(19) United States
(12) Patent Application Publication
(10) Pub. No.: US 2013/0267853 A1
(43) Pub. Date: Oct. 10, 2013

(54) ULTRASOUND DEVICE, AND ASSOCIATED CABLE ASSEMBLY

(71) Applicant: RESEARCH TRIANGLE INSTITUTE, Research Triangle Park, NC (US)

(72) Inventors: David Dausch, Raleigh, NC (US); James Carlson, Durham, NC (US); Kristin Hedgepath Gilchrist, Durham, NC (US); Stephen Hall, Apex, NC (US)

(21) Appl. No.: 13/907,010

(22) Filed: May 31, 2013

Related U.S. Application Data
(63) Continuation of application No. PCT/US2011/062665, filed on Nov. 30, 2011.
(60) Provisional application No. 61/419,507, filed on Dec. 3, 2010.

(51) Int. Cl. A61B 8/00

(52) U.S. Cl. CPC A61B 8/4494; A61B 8/4488; A61B 8/445
USPC 600/466; 600/459

(57) ABSTRACT

An ultrasound device including an ultrasonic transducer device having a plurality of transducer elements forming a transducer array is provided. Each transducer element includes a piezoelectric material disposed between a first electrode and a second electrode. One of the first and second electrodes is a ground electrode and the other of the first and second electrodes is a signal electrode. The ultrasound device further includes a cable assembly having a plurality of connective signal elements and a plurality of connective ground elements extending in substantially parallel relation therealong. Each connective element is configured to form an electrically-conductive engagement with respective ones of the signal electrodes and the ground electrodes of the transducer elements in the transducer array. The connective ground elements are alternatingly disposed with the connective signal elements across the cable assembly, to provide shielding between the connective signal elements.

### BACKGROUND OF THE DISCLOSURE

1. Field of the Disclosure

Aspects of the present disclosure relate to ultrasonic transducers, and, more particularly, to an ultrasound apparatus having a cable assembly for forming a connection with a piezoelectric micromachined ultrasonic transducer housed in a catheter.

2. Description of Related Art

Some micromachined ultrasonic transducers (MUTs) may be configured, for example, as a piezoelectric micromachined ultrasonic transducer (pMUT) as disclosed in U.S. Pat. No. 7,449,821 assigned to Research Triangle Institute, also the assignee of the present disclosure, which is also incorporated herein in its entirety by reference.

The formation of a pMUT device, such as the pMUT device defining an air-backed cavity as disclosed in U.S. Pat. No. 7,449,821, may involve the formation of an electrically-conductive connection between the first electrode (i.e., the bottom electrode) of the transducer device, wherein the first electrode is disposed on the front side of the substrate opposite to the air-backed cavity of the pMUT device, and the conformal metal layer(s) applied to the air-backed cavity for providing subsequent connectivity, for example, to an integrated circuit (“IC”) or a flex cable.

In some instances, one or more pMUTs, for example, arranged in a transducer array, may be incorporated into the end of an elongate catheter or endoscope. In those instances, for a forward-looking arrangement, the transducer array of pMUT devices must be arranged such that the plane of the piezoelectric element of each pMUT device is disposed perpendicularly to the axis of the catheter/endoscope. This configuration may thus limit the lateral space about the transducer array, between the transducer array and the catheter wall, through which signal connections may be established with the front side of the substrate. Further, directing such signal connections laterally to the transducer array to the front side thereof, may undesirably and adversely affect the diameter of the catheter (i.e., a larger diameter catheter may undesirably be required in order to accommodate the signal connections passing about the transducer array).

Where the transducer array is a one-dimensional (1D) array, external signal connections to the pMUT devices may be accomplished by way of a flex cable spanning the series of pMUT devices in the transducer array so as to be in electrical engagement with (i.e., bonded to) each pMUT device via the conformal metal layer thereof. For instance, As shown in FIG. 1, in one exemplary 1D transducer array 100 (e.g., 1×64 elements), pMUT devices forming the array elements 120 may be attached directly to a flex cable 140, with the flex cable 140 including one electrically-conductive signal lead per pMUT device, plus a ground lead. For a forward-looking transducer array, the flex cable 140 is bent about the opposing ends of the transducer array such that the flex cable 140 can be routed through the lumen of the catheter/endoscope which, in one instance, may comprise an ultrasound probe. However, for a forward-looking transducer array in a relatively small catheter/endoscope, such an arrangement may be difficult to implement due to the severe bend requirement for the flex cable (i.e., about 90 degrees), which may also be compounded by the number of conductors comprising the flex cable and the engagement of the electrically-conductive signal leads to the pMUT devices (also about a bend of about 90 degrees), in order for the transducer array to be disposed within the lumen of the catheter/endoscope.

Further, for a forward-looking two-dimensional (2D) transducer array, signal interconnection with the individual pMUT devices may also be difficult. That is, for an exemplary 2D transducer array (e.g. 14×14 to 40×40 elements), there may be many more required signal interconnections with the pMUT devices, as compared to a 1D transducer array. As such, more wires and/or multilayer flex cable assemblies may be required to interconnect with all of the pMUT devices in the transducer array. However, as the number of wires and/or flex cable assemblies increases, the more difficult it becomes to bend the larger amount of signal interconnections about the ends of the transducer device to achieve the 90 degree bend required to integrate the transducer array into a catheter/endoscope. In addition, the pitch or distance between adjacent pMUT devices may be limited due to the required number of wires/conductors. Accordingly, such limitations may undesirably limit the minimum size (i.e., diameter) of the catheter/endoscope that can readily be achieved.

Co-pending U.S. patent application Ser. No. 61/329,258 (Methods for Forming a Connection with a Micro machined Ultrasonic Transducer, and Associated Apparatuses; filed Apr. 29, 2010, and assigned to Research Triangle Institute, also the assignee of the present application), discloses improved methods of forming an electrically-conductive connection between a pMUT device and, for example, an integrated circuit (“IC”), a flex cable, or a cable assembly, wherein individual signal leads extend parallel to the operational direction of the transducer array or perpendicularly to the transducer array face to engage the respective pMUT devices in the transducer array (see generally, e.g., FIG. 2). Furthermore, the '258 application discloses that additional signal processing integrated circuits (IC's) can be integrated between the transducer array and the corresponding connective elements, thereby increasing the dimension of the transducer/connective element stack in a longitudinal direction of the disposition thereof in the catheter, but not increasing the lateral spacing around the transducer array, thus facilitating the configuration of the catheter to achieve a minimal diameter for a forward-looking transducer array configuration.

In the case of side- or lateral-looking transducer arrays, the transducer array is arranged such that the plane of the piezoelectric element of each transducer device is disposed in parallel to the axis of the catheter/endoscope. In such instances, there is relatively more lateral space about the transducer array, between the transducer array and the catheter wall, along the length of the transducer array, which may be used to attach connective elements thereto. However, the space between the back side of the transducer array and the catheter wall may be limited, particularly, for example, in catheters having an inner diameter of about 3 mm or less. Further, the previously-noted thicker stacks placed in a transducer arrangement, as illustrated in FIG. 2 and including a transducer array, signal processing IC's and connective elements, may not necessarily be feasible in instances of the limited catheter inner diameter. Such a configuration may also undesirably impart mechanical stresses to the signal lead (which must be bent about 90 degrees to be routed from the transducer and along the catheter) and/or transducer array interface due to the thickness of the transducer/IC stack and the limited space available across the catheter diameter. One particular example of a prior art side-looking ultrasound catheter transducer is shown in FIG. 3, wherein a piezoelectric element 200 may be attached to a flex cable 210 using conductive epoxy 220. A top electrode 230 and matching layer 240 may then be deposited on the piezoelectric element 200, and the structure is then diced using a saw, wherein the cuts extend down to the flex cable 210 in order to form the elements of the transducer array 250. An acoustic backing 260 may then be applied to the back of the flex cable 210. However, such a configuration may be limited with respect to the number of transducer elements that can be practically implemented due, for instance to the resolution limit of the signal traces of the flex cable. For example, for a 3 mm catheter, only 16 traces with 100 μm pitch (plus ground strips on each side) may fit laterally within the lumen of the catheter. As such, an appropriate flex cable, such as a Siemens Akuna flex cable with 64 elements, may undesirably have to be folded into 4 layers of 16 traces each (plus grounds) to connect all of the elements of a 64 element transducer array. Further, for 2D transducer arrays, high element counts (e.g., 196 to 1,600 elements) may require multilayer flex cabling for attachment and interconnection of all transducer elements, further increasing cost and complexity of the flex cabling. Multilayer flex cable could require up to 16 levels to connect all transducer elements due to limitations, for example, related to the pitch of conductor traces and interlevel vias in the flex cable (i.e., typically having a minimum of 100 μm pitch or more, depending on the number of levels). Further, for 2D arrays, a flex cable containing several hundred conductors may be too large in dimension (i.e., too wide and/or too thick) to fit within a 3 mm diameter catheter. A multiple level flex cable may thus be undesirably expensive, difficult (or impossible) to manufacture, and may not be robust due to a relatively high probability of short circuits in light of the increased number of metal levels and vias. Other disadvantages of multilayer flex cabling may include higher conductor impedance, higher insertion loss, greater cross coupling between element traces, and higher shunt-to-ground capacitance which may reduce penetration depth compared to coaxial cabling (though typical coaxial cabling cannot be made with sufficiently fine pitch to be used in such catheter applications). Flex cabling may also be typically limited to segments of approximately 1 foot in length. Thus for a catheter that is 3 feet in total length, multiple flex cable segments must be serially connected in order to complete the electrical connection through the entire catheter, thereby undesirably increasing complexity and cost of assembly.

Thus, there exists a need in the ultrasonic transducer art, particularly with respect to a piezoelectric micromachined ultrasound transducer (“pMUT”), whether having an air-backed cavity or not, for improved methods of forming an electrically-conductive connection between the pMUT device and, for example, an integrated circuit (“IC”) and/or corresponding connective elements. More particularly, it would be desirable for such an electrically-conductive connection with the pMUT device to be configured to avoid bending of the flex cable/wiring about the pMUT device upon integration thereof in the tip of a probe/catheter/endoscope used, for example, in cardiovascular devices, intravascular and intracardiac ultrasound devices, and laparoscopic surgery devices. Furthermore, it would be desirable to provide a method for forming electrical connections with a transducer array having a relatively higher transducer element count/density that is cost efficient (i.e., relatively low cost) and relatively manufacturable. Such solutions should desirably be effective for 2D transducer arrays, particularly 2D pMUT transducer arrays, but should also be applicable to 1D transducer arrays, in forward-looking and/or side looking arrangements, and should desirably allow greater scalability in the size of the probe/catheter/endoscope having such transducer arrays integrated therein.

### BRIEF SUMMARY OF THE DISCLOSURE

The above and other needs are met by aspects of the present disclosure, wherein one such aspect relates to an ultrasound device comprising an ultrasonic transducer device comprising a plurality of transducer elements forming a transducer array. Each transducer element comprises a piezoelectric material disposed between a first electrode and a second electrode. One of the first and second electrodes comprises a ground electrode and the other of the first and second electrodes comprises a signal electrode. The ultrasound device further includes a cable assembly comprising a plurality of connective signal elements and a plurality of connective ground elements extending in substantially parallel relation therealong. Each connective element is configured to form an electrically-conductive engagement with respective ones of the signal electrodes and the ground electrodes of the transducer elements in the transducer array. The connective ground elements are configured to be alternatingly disposed with the connective signal elements across the cable assembly, to provide shielding between the connective signal elements.

Yet another aspect of the present disclosure provides an ultrasound device comprising an ultrasonic transducer device comprising a plurality of transducer elements forming a transducer array. Each transducer element comprises a piezoelectric material disposed between a first electrode and a second electrode. One of the first and second electrodes comprises a ground electrode and the other of the first and second electrodes comprises a signal electrode. The ultrasound device further comprises a catheter member having a distal end and defining a longitudinally-extending lumen, wherein the lumen is configured to receive the ultrasonic transducer device about the distal end. The ultrasound device further comprises a cable assembly comprising a plurality of connective signal elements and a plurality of connective ground elements extending in substantially parallel relation therealong. Each connective element is configured to form an electrically-conductive engagement with respective ones of the signal electrodes and the ground electrodes of the transducer elements in the transducer array. The connective ground elements are configured to be alternatingly disposed with the connective signal elements across the cable assembly such that the connective ground elements provide shielding between the connective signal elements.

Aspects of the present disclosure thus address the identified needs and provide other advantages as otherwise detailed herein.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIGS. 1 and 2 schematically illustrate a prior art arrangements for forming a connection with a forward-looking transducer apparatus disposed in a lumen;

FIG. 3 schematically illustrates a prior art arrangement for forming a connection with a side-looking transducer apparatus disposed in a lumen;

FIG. 4 schematically illustrates a cross-sectional view of an exemplary piezoelectric ultrasonic transducer device, having both ground and signal electrodes disposed about the back side of the substrate, according to one aspect of the disclosure;

FIG. 5 is a schematic end view of a pMUT device (array) having ground electrodes arranged about the periphery thereof, with signal electrodes within the periphery, according to one aspect of the disclosure;

FIG. 6 is a schematic perspective view of an arrangement for assembling connective signal elements with a connection support substrate, according to one aspect of the disclosure;

FIG. 7 is a schematic end view of the arrangement illustrated in FIG. 6;

FIG. 8 is a schematic cross-sectional view of a cable assembly configured to form a connection, for example, with the device shown in FIG. 5, according to one aspect of the disclosure;

FIG. 9 is a schematic end view of a pMUT device (array) having ground electrodes interstitially disposed with respect to signal electrodes, according to one aspect of the disclosure;

FIG. 10 is a schematic perspective view of an arrangement for foil ling a connection with a pMUT device, according to one aspect of the disclosure;

FIG. 11 is a schematic cross-sectional view of a cable assembly configured to form a connection, for example, with the device shown in FIG. 9, according to one aspect of the disclosure;

FIG. 12 schematically illustrates a top view of an interposer device arrangement for forming a connection with a side-looking two-dimensional pMUT device, according to another aspect of the disclosure;

FIG. 13 schematically illustrates an exemplary pMUT device having connective signal and ground elements engaged therewith, according to one aspect of the disclosure;

FIG. 14 schematically illustrates a forward-looking ultrasound device, according to one aspect of the present disclosure;

FIG. 15 schematically illustrates a side-looking ultrasound device, according to one aspect of the present disclosure;

FIG. 16 is a schematic plan view of an arrangement for forming a connection with a pMUT device, according to still another aspect of the disclosure;

FIG. 17 is a schematic partial cut away view of the arrangement illustrated in FIG. 16;

FIG. 18A is a schematic cross-sectional side view of an arrangement for forming a connection with a forward-looking two-dimensional piezoelectric micromachined ultrasonic transducer device, according to a further aspect of the disclosure;

FIG. 18B is a schematic cross-sectional side view of another arrangement for forming a connection with a forward-looking two-dimensional piezoelectric micromachined ultrasonic transducer device, according to a further aspect of the disclosure;

FIG. 19 is a schematic cross-sectional side view of an arrangement for forming a connection with a forward-looking two-dimensional piezoelectric micromachined ultrasonic transducer device, according to still another aspect of the disclosure;

FIG. 20 is a schematic cross-sectional side view of an arrangement for forming a connection with a forward-looking two-dimensional piezoelectric micromachined ultrasonic transducer device, according to still yet another aspect of the disclosure;

FIGS. 21 and 22 are schematic end views of pMUT devices (arrays) having ground electrodes disposed about a periphery thereof with respect to signal electrodes, according to various aspects of the disclosure;

FIGS. 23 and 24 are schematic cross-sectional side views of arrangements for forming a connection with a forward-looking two-dimensional piezoelectric micromachined ultrasonic transducer device, according to various aspects of the disclosure;

FIG. 25A schematically illustrates a forward-looking ultrasound device, according to one aspect of the present disclosure;

FIG. 25B schematically illustrates a side-looking ultrasound device, according to one aspect of the present disclosure;

FIG. 26 schematically illustrates an exemplary interposer device, according to one aspect of the present disclosure;

FIG. 27 schematically illustrates an exemplary interposer device incorporated into a circuit board assembly, according to one aspect of the present disclosure;

FIG. 28 schematically illustrates a exemplary component layout for an ultrasound device including associated cable assembly and termination element, according to one aspect of the present disclosure;

FIGS. 29 and 30 schematically illustrate an exemplary pMUT device (array) engaged with a connection support substrate having associated connective signal and ground elements, according to one aspect of the present disclosure;

FIGS. 31 and 32 schematically illustrate an exemplary termination element, such as a printed circuit board, engaged with a cable assembly having associated connective signal and ground elements, according to one aspect of the present disclosure; and

FIG. 33 schematically illustrates the distal end of an exemplary catheter assembly having a transducer array, a connection support substrate, and connective signal and ground elements disposed in the catheter housing, according to one aspect of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as limited to the aspects set forth herein; rather, these aspects are provided so that this disclosure will satisfy applicable legal requirements. Like numbers refer to like elements throughout.

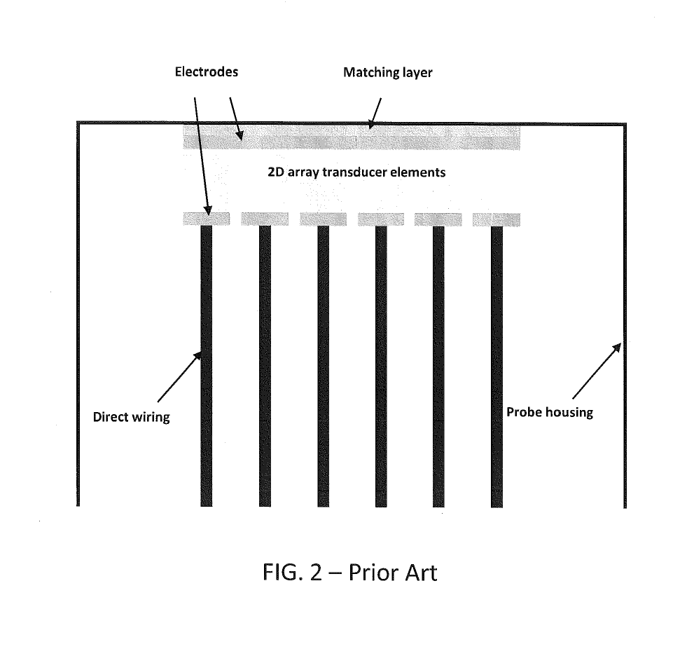

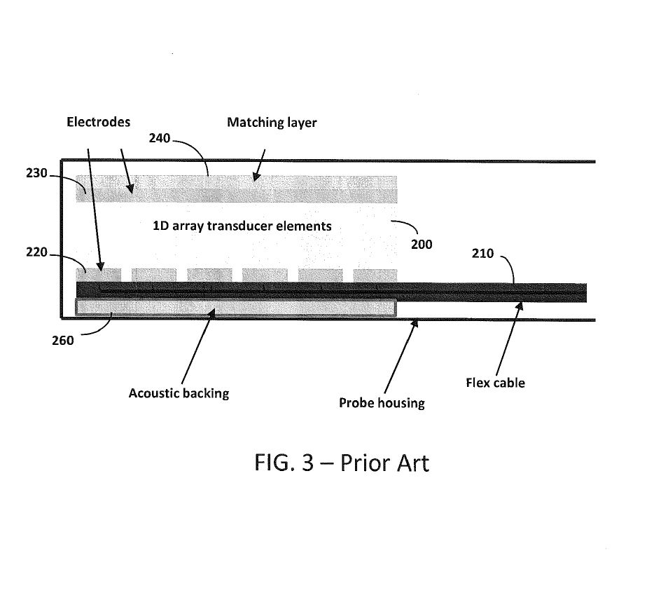

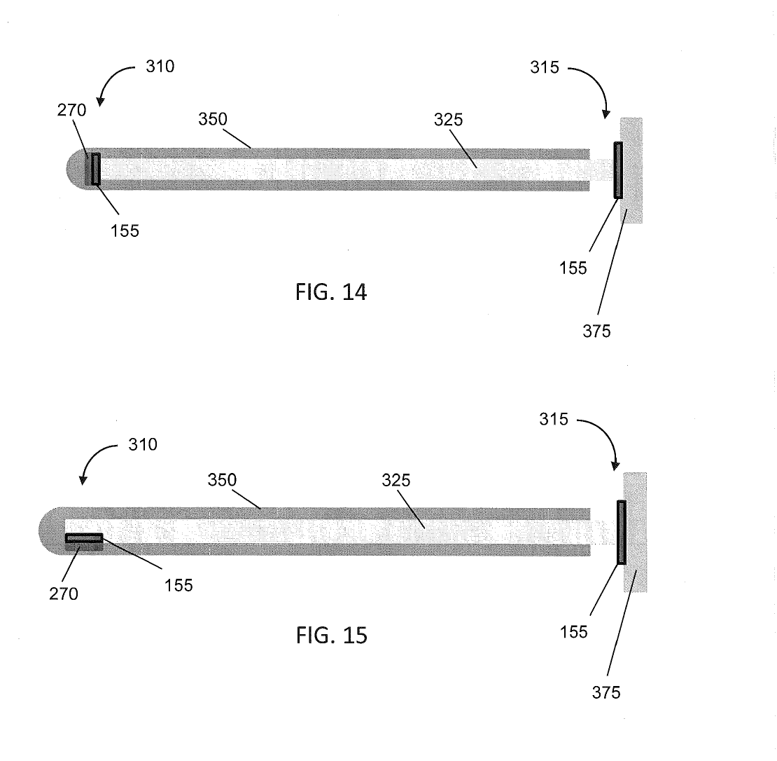

Aspects of the present disclosure are generally applicable to ultrasonic transducers, though particular aspects are particularly directed to a piezoelectric micromachined ultrasound transducer (“pMUT”) having an air-backed cavity. More particularly, aspects of the present disclosure are directed to methods of forming an electrically-conductive connection between a pMUT device and, for example, an integrated circuit (“IC”) and/or corresponding connective elements, whereby individual signal and ground leads may extend parallel to the operational direction of the transducer array to engage the respective pMUT devices in the transducer array (see generally, e.g., FIG. 2). The pMUT device may be disposed within a catheter member 350 having a distal end or tip 310 (see, e.g., FIGS. 14 and 15). The catheter member 350 may further define a longitudinally-extending lumen configured to receive the pMUT device about the distal end 310. The pMUT device may further comprise a cable assembly 325 comprising connective elements, and having one or more connection support substrates 155 disposed about the distal end 310 and proximal end 315 of the catheter.

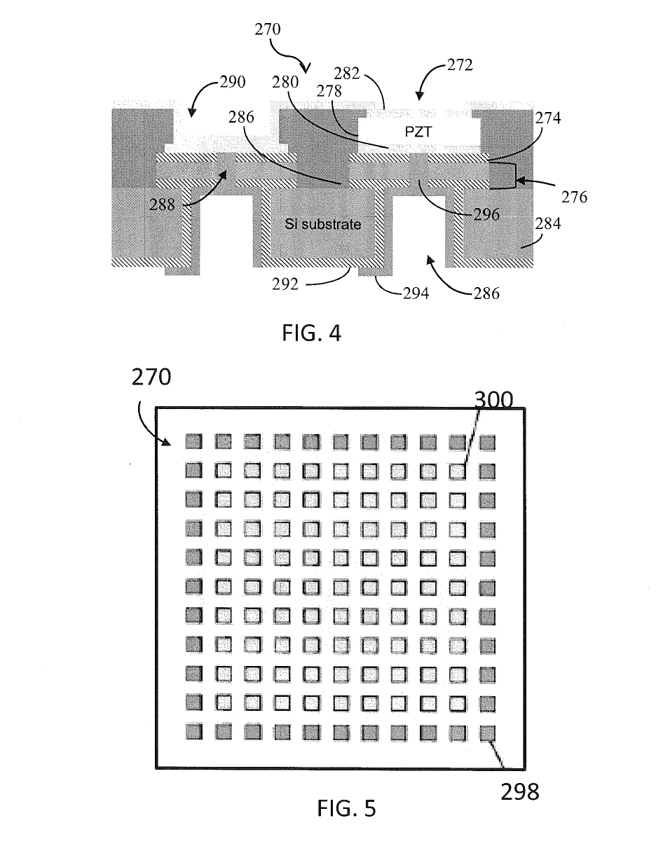

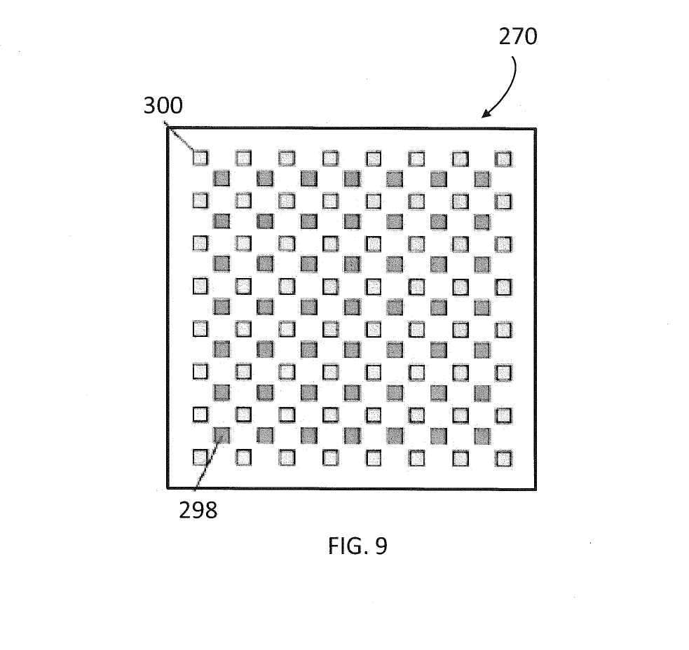

In such aspects, a representative pMUT or ultrasonic transducer device 270 that may be implemented in both 1D and 2D transducer arrays, may generally comprise a plurality of transducer elements forming a transducer array, wherein each transducer element 272 comprises a piezoelectric material disposed between a first electrode and a second electrode, and wherein one of the first and second electrodes comprises a ground electrode and the other of the first and second electrodes comprising a signal electrode. More particularly, as shown, for example, in FIG. 4, a transducer element 272 may be disposed on a dielectric layer 274 of a device substrate 276, wherein the transducer element 272 includes a piezoelectric material 278 disposed between a first electrode 280 and a second electrode 282. The primary substrate 284 defines a first via 286 extending to the device substrate 276, while the device substrate further defines a second via 288 extending therethrough to the first electrode 280. In some instances, the first and second via 286, 288 arrangement may extend to the first electrode 280, which is engaged with the second electrode 282 (shown generally at element 290), thereby connecting the second electrode 282 to the back side of the substrate. Such an arrangement may be applied, for example, as a back side ground pad or ground electrode for the pMUT device 270. In such instances, the first and second vias 286, 288 may have a conformal insulating layer 292 deposited therein, before the first and second vias 286, 288 are substantially filled with first and second conductive materials, respectively 294, 296. In some aspects, the first and second electrode engagement 290 may be disposed, as necessary or desired about a pMUT array. In some instances, such an arrangement 290 may be disposed about the periphery of the pMUT array incorporating the pMUT device structure 270 (see, e.g., FIG. 5), or in interstices between adjacent pMUT device structures 270 within a pMUT array (see, e.g., FIG. 9). Such pMUT devices 270 are disclosed, for example, in U.S. Provisional Patent Application No. 61/299,514 (“Methods for Forming a Micromachined Ultrasonic Transducer, and Associated Apparatuses”), assigned to Research Triangle Institute, and which is incorporated herein in its entirety by reference.

Particular materials that can be implemented for the piezoelectric material 278 include, for example, ceramics including ZnO, AlN, LiNbO₄, lead antimony stannate, lead magnesium tantalate, lead nickel tantalate, titanates, tungstates, zirconates, or niobates of lead, barium, bismuth, or strontium, including lead zirconate titanate (Pb(ZrₓTi₁₋ₓ)O₃ (PZT)), lead lanthanum zirconate titanate (PLZT), lead niobium zirconate titanate (PNZT), BaTiO₃, SrTiO₃, lead magnesium niobate, lead nickel niobate, lead manganese niobate, lead zinc niobate, lead titanate. Piezoelectric polymer materials such as polyvinylidene fluoride (PVDF), polyvinylidene fluoride-trifluoroethylene (PVDF-TrFE), or polyvinylidene fluoride-tetrafluoroethylene (PVDF-TFE) can also be used.

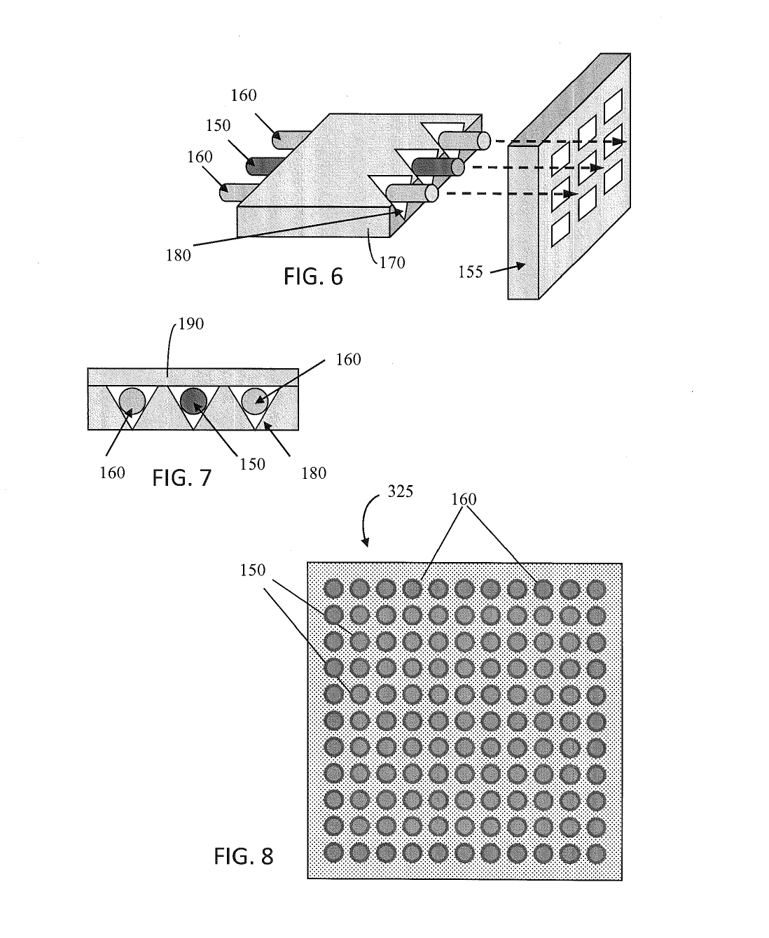

One method of forming an electrically-conductive connection with one configuration of a pMUT device 270, in the form of a two-dimensional forward-looking pMUT array, is schematically illustrated, for example, in FIGS. 6-8. In such instances, the pMUT device (array) 270 may incorporate a plurality of pMUT elements 272, with the pMUT array having ground pads or electrodes 298 (e.g., as associated with the first and second electrode engagement 290 of the pMUT device 270, as shown in FIG. 4) arranged about the periphery of the array such that the signal pads or electrodes 300 (see, e.g., FIG. 5; as associated with transducer element 272 of the pMUT device 270) are arranged within the periphery, for example, in regular rows. In such aspects, signal leads (connective elements) and ground leads (connective elements) extending parallel to the operational direction of the transducer array may be configured to form electrically conductive engagements with the respective ground and signal electrodes 298, 300 of the pMUT elements 272 within the array, wherein, in some instances, one or more of the ground electrodes 298 may be common to more than one of the pMUT elements 272 within the array 270.

According to one aspect, as shown in FIGS. 6 and 7, a 2D array of connective elements (i.e., wires) may include connective signal elements 150 and connective ground elements 160, each of which may or may not be coated with an insulator layer. In one instance, it may be desirable that at least the connective signal elements 150 each be coated with an insulator layer. In order to form the electrically-conductive engagement between the connective signal and ground elements 150, 160 and the respective signal and ground electrodes 300, 298 of the pMUT device (array) 270, the connective signal and ground elements 150, 160 may first be arranged such that first ends thereof are engaged with and supported by a connection support substrate 155. Such an engagement may be accomplished, for instance, using a guide substrate 170 defining a plurality of parallel, spaced-apart channels 180 extending across the width thereof (and along the length of the guide substrate 170), wherein the lateral pitch of the channels 180 generally corresponds to the lateral pitch of the connective elements 150, 160 of the pMUT device 270. Once the connective elements 150, 160 are inserted into the respective channels 180, so as to extend longitudinally outward thereof, a retaining member 190 may be removably applied over the channels 180 so as to retain the connective elements 150, 160 within the channels 180. Once prepared, the guide substrate 170 may be disposed adjacent to the intended connection support substrate 155 (i.e., using micropositioners), and the connective elements 150, 160 directed along the channels 180 to engage the connection support substrate 155 for securement therein. The connection support substrate 155 may be, for example, a silicon substrate with through-holes etched using deep reactive ion etching (DRIE) to provide straight (i.e., substantially vertical or otherwise substantially perpendicular to the plane of the substrate) sidewalls in the through-holes, which may facilitate high density arrays of holes (i.e. without lateral angled sidewalls created by a wet etching process such as KOH etching). Once engaged with the connection support substrate 155, the connective elements 150, 160 may be fixed in the through-holes of the connection support substrate 155 using an adhesive material such as, for instance, an insulating epoxy. The free or unengaged surface of the connection support substrate 155 may then be polished or otherwise planarized to facilitate subsequent bonding to the pMUT device 270. Such a process for engaging the connective elements 150, 160 with a connection support substrate 155 is disclosed, for example, in U.S. patent application Ser. No. 61/329,258 (“Methods for Forming a Connection with a Micromachined Ultrasonic Transducer, and Associated Apparatuses”), previously been incorporated by reference, and which discloses other methods for engaging connective elements with a connection support substrate, as well as forming an electrically-conductive engagement between the connective elements and a pMUT device, a transducer array, or an intermediate device such as an interposer.

In this regard, the connection support substrate 155, having the connective signal and ground elements 150, 160 engaged therewith, may be configured to engage the pMUT device 270 about the signal and ground electrodes 300, 298, so as to provide, for example, an appropriate pitch or spacing of the connective signal and ground elements 150, 160 in correspondence with the pMUT device 270, as well as mechanical support for the direct electrically-conductive engagement between the connective signal and ground elements 150, 160 and the respective signal and ground electrodes 300, 298. As shown in FIG. 8, the connective elements 150, 160 may thus be assembled with the connection support substrate 155 such that the connective ground elements 160 are disposed about the periphery of the connection support substrate 155 so as to correspond to the ground electrodes 298 and to facilitate the formation of the electrically-conductive connection therewith. The conductive signal elements 150 are thus disposed within the periphery of the connection support substrate 155 so as to correspond to the signal electrodes 300 and to facilitate the formation of the electrically-conductive connection therewith.

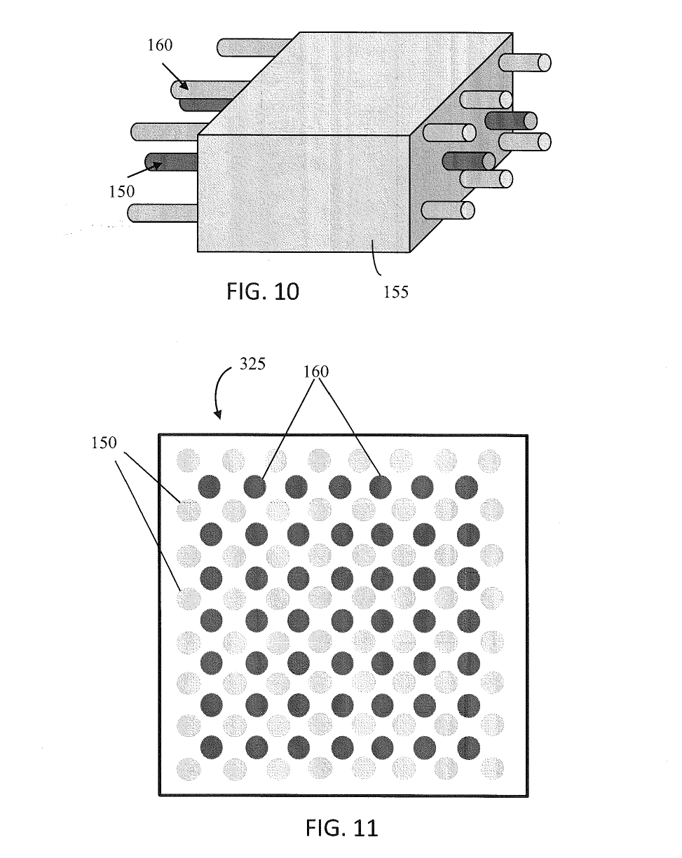

Further aspects of the present disclosure may be directed to a method of forming an electrically-conductive connection with another configuration of a pMUT device 270, in the form in of a two-dimensional forward-looking pMUT array, is schematically illustrated, for example, in FIGS. 9-11. In such instances, the pMUT device (array) 270 may incorporate a plurality of pMUT elements 272, with the pMUT array having the signal pads or electrodes 300 (see, e.g., FIG. 5; as associated with transducer element 272 of the pMUT device 270) arranged, for example, in regular rows, with the ground pads or electrodes 298 (e.g., as associated with the first and second electrode engagement 290 of the pMUT device 270, as shown in FIG. 4) arranged in the interstices of the signal electrodes 300 of the array (see, e.g., FIG. 9). According to such an arrangement, the ground electrodes 298 may be considered interspersed within the regular rows of signal electrodes 300, or the rows of ground electrodes 298 may be laterally shifted by about half of the pitch or spacing between signal electrodes 300 such that alternating rows of signal electrodes 300 and ground electrodes 298 are staggered with respect to each other. In such aspects, signal leads (connective elements) and ground leads (connective elements) extending parallel to the operational direction of the transducer array may be correspondingly configured (see, e.g., FIGS. 10 and 11) to form electrically conductive engagements with the respective ground and signal electrodes 298, 300 of the pMUT elements 272 within the array, wherein, in some instances, one or more of the ground electrodes 298 may be common to more than one of the pMUT elements 272 within the array 270. As such, one skilled in the art will appreciate that various arrangements of the connective elements 150, 160 may be required to form the electrically-conductive engagements with the various arrangements of pMUT elements 272 within the pMUT device (array) 270.

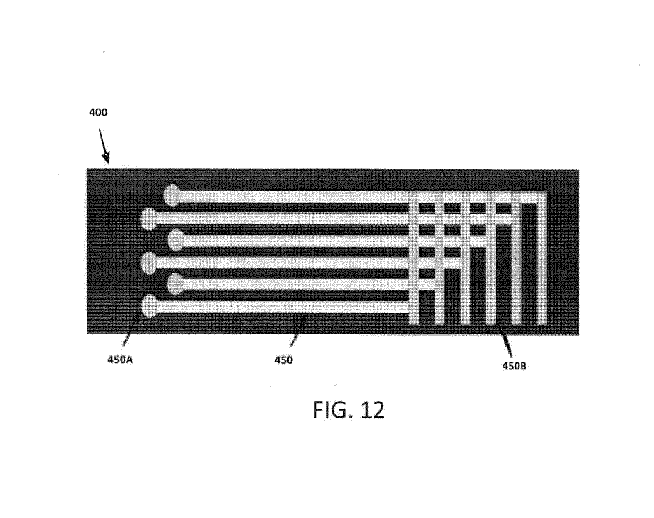

According to some aspects, the connective elements 150, 160 may be electrically-engaged with a side- or lateral-looking transducer array. A representative ultrasound device implementing a side- or lateral-looking transducer array is disclosed, for example, in U.S. Provisional Patent Application No. 61/419,534 (“Method for Fanning an Ultrasound Device, and Associated Apparatus”), filed concurrently herewith, and which is incorporated herein in its entirety by reference. In such instances, particularly when the pMUT device (array) is disposed within a catheter, the connective elements 150, 160 extend along the catheter, and are required to engage the transducer array having the ground and signal electrodes 298, 300 of the pMUT elements 272 perpendicularly disposed with respect to the longitudinal axis of the catheter. In such instances, the connection support substrate 155 may be configured to facilitate navigation of the change in direction (i.e., having the channels extending therethrough at an angle of about 90 degrees with respect to the longitudinal axis of the catheter). In other instances, the connective elements 150, 160 may be configured to engage an interposer device 400, as shown, for example, in FIG. 12, configured to receive, engage and support an ultrasonic transducer apparatus (array—not shown) such that a device plane of the ultrasonic transducer apparatus extends substantially parallel to the interposer device 400. In some instances, the interposer device 400 also includes at least two conductors 450 extending therealong (i.e., through the interposer device 400 or along a surface thereof), wherein the conductors 450 each have opposed first and second ends 450A, 450B. One of the ends 450B may be configured to engage the respective signal and ground electrodes 298, 300, in some instances, via wirebond pads such as, for example in a wire bonding process; while the other of the ends 450A may be configured to engage the connective signal and ground elements 150, 160, whether directly or via a connection support substrate.

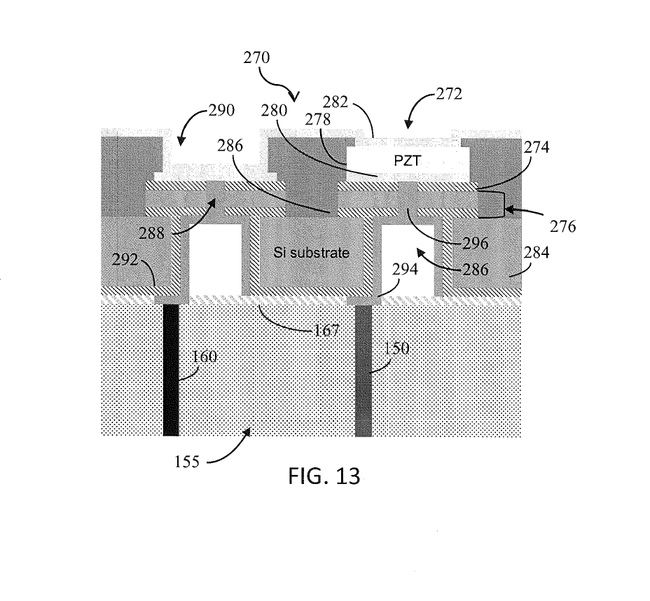

As shown in FIG. 13, one end of the cable assembly 325, incorporating the connection support substrate 155 and the connective signal and ground elements 150, 160, may be polished or otherwise planarized (i.e., perpendicularly to the longitudinal axis) so as to provide a planar surface for bonding, with an appropriate bonding material 167, the same to the pMUT device (array) 270. Thus, the cable assembly 325 may be bonded or otherwise engaged with the pMUT device 270 in a number of manners, for example, according to methods and configurations described, for example, in U.S. patent application Ser. No. 61/329,258 (“Methods for Forming a Connection with a Micromachined Ultrasonic Transducer, and Associated Apparatuses”), previously incorporated by reference. In general, connective elements 150, 160 may be assembled into connection support substrates and bonded to transducer arrays/pMUT devices, interposers or other termination element, as disclosed, for example, in U.S. patent application Ser. No. 61/329,258 (“Methods for Forming a Connection with a Micromachined Ultrasonic Transducer, and Associated Apparatuses”) assigned to Research Triangle Institute (also the assignee of the present disclosure), and previously incorporated herein by reference. In some instances, as shown, for example, in FIGS. 14 and 15, the cable assembly 325, incorporating the connection support substrate 155 and the connective signal and ground elements 150, 160, may be terminated at both ends 310, 315 thereof with a connection support substrate, interposer, or other termination element. More particularly, one end 310 is configured to engage the pMUT device (array) 270, whether directly via a connection support substrate or via an interposer device, while the opposing end 315 extends along and/or outwardly of the catheter 350, away from the tip thereof, to engage a connection support substrate, interposer, circuit board (i.e., associated with a computer device), semiconductor package, or other termination element (see, generally, element 375) configured to provide external connectivity between the pMUT device (array) 270 disposed in the catheter 350 and, for example, an external ultrasound system or other image display device. The connective elements 150, 160 may be individually assembled at both ends of the cable assembly 325 such that the connective elements 150, 160 may be mapped or otherwise tracked with respect to connectivity to the transducer elements 272 within the array 270, such that, for instance, the locations of the transducer elements within the transducer array can be identified and controlled by the appropriate electronic channels in the external ultrasound system.

In light of the aspects previously disclosed, with respect to forming electrically-conductive engagements with the pMUT elements 272 in the pMUT array 270, some aspects of the present disclosure are further directed to a cable assembly 325 comprising a plurality of connective signal elements 150 and a plurality of connective ground elements 160 extending in substantially parallel relation along the cable assembly 325, with each being configured to form an electrically-conductive engagement with respective ones of the signal electrodes 300 and the ground electrodes 298 of the transducer elements 272 in the transducer array 270. More particularly, in some aspects, the connective ground elements 160 are configured to be alternatingly disposed (i.e., whether constructively or actually) with the connective signal elements 150 across the cable assembly 325, so as to provide shielding between the connective signal elements 150.

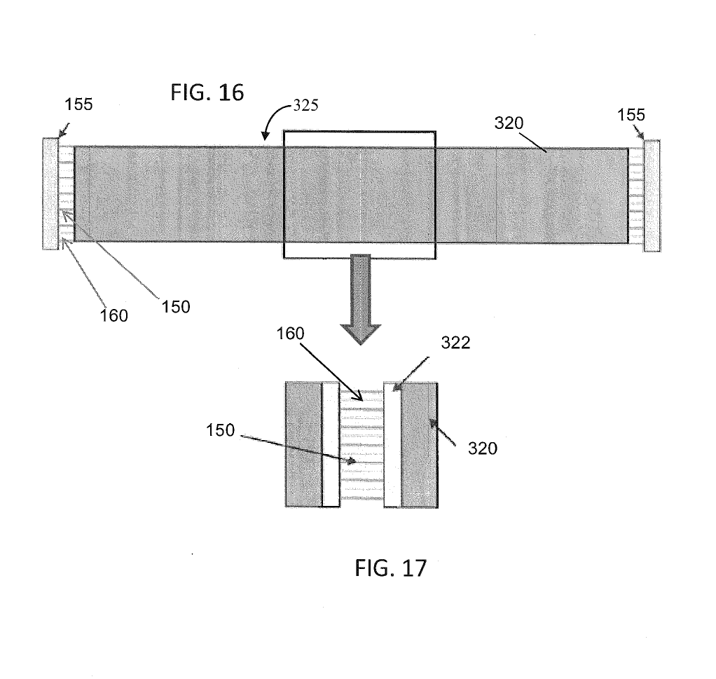

In some instances, as shown, for example, in FIGS. 16 and 17, the cable assembly 325 may be configured to provide alternatingly disposed connective ground elements 160 and connective signal elements 150 (i.e., in actual correspondence with the configuration of the signal and ground electrodes 300, 298 of the transducer array 270). That is, the connective ground elements 160 may be interspersed among the connective signal elements 150, or otherwise be disposed between two or more connective signal elements 150 (i.e., in interstices between adjacent connective signal elements 150). The alternatingly disposed connective ground elements 160 and connective signal elements 150 (see, e.g., FIG. 11) may further be engaged at opposing ends 310, 315 with connection support substrates 155, wherein at one such end 310, a connection support substrate 155 may be engaged with the transducer array 270, in some instances with an interposer device disposed therebetween, while the opposing end 315 may have one or more connection support substrates 155 engaged with an interposer, circuit board, semiconductor package, or other termination element, as previously disclosed. By actually alternating the connective ground elements 160 and the connective signal elements 150, the connective ground elements 160 may function as a shield or ground between the connective signal elements 150 in order, for example, to reduce cross-talk between the connective signal elements 150 with respect to signals from the transducer elements 272. The connective elements 150, 160, comprised of, for example, relatively fine gauge wire (e.g., insulated magnet wire having a diameter of between about 40 AWG and about 50 AWG), may be individually engaged with the connection support substrates 155 between opposing ends so as to provide a registration with respect to the connective ground and signal elements 150, 160. In some instances, for example, a color indicia scheme may be implemented to distinguish the connective ground and signal elements 150, 160.

In some instances, the connective elements 150, 160 of the cable assembly 325 may be encapsulated with a dielectric material, such as, for example, a conformal dielectric coating 320, to seal and bundle the connective elements 150, 160 to form the cable assembly 325. In other instances, the connective elements 150, 160 may be wrapped with an outer covering, such as, for example, a shrinkable tubing, extending therealong so as to provide a flexible but robust cable assembly 325. In further instances, additional shielding for the connective elements 150, 160 may be provided, for example, by a conductive film, such as, for example, a metal foil material 322 (e.g., MYLAR®), wrapped about the connective elements 150, 160. The dielectric coating 320 may be applied to cover the conductive film 322, such that the conductive film 322 is disposed between the connective elements 150, 160 and the dielectric coating 320. In other instances (not shown), a conductive film may be wrapped about the dielectric material 320, so as to be disposed between the catheter member 350 and the dielectric material 320 encapsulating the connective signal and ground elements 150, 160. In either instance, the conductive film 322 may provide additional shielding for at least the connective signal elements 150, 160. Still in other instances, additional shielding may be molded or otherwise incorporated into the catheter member 350 such as, for example, a metal braid (not shown) molded into the catheter member 350.

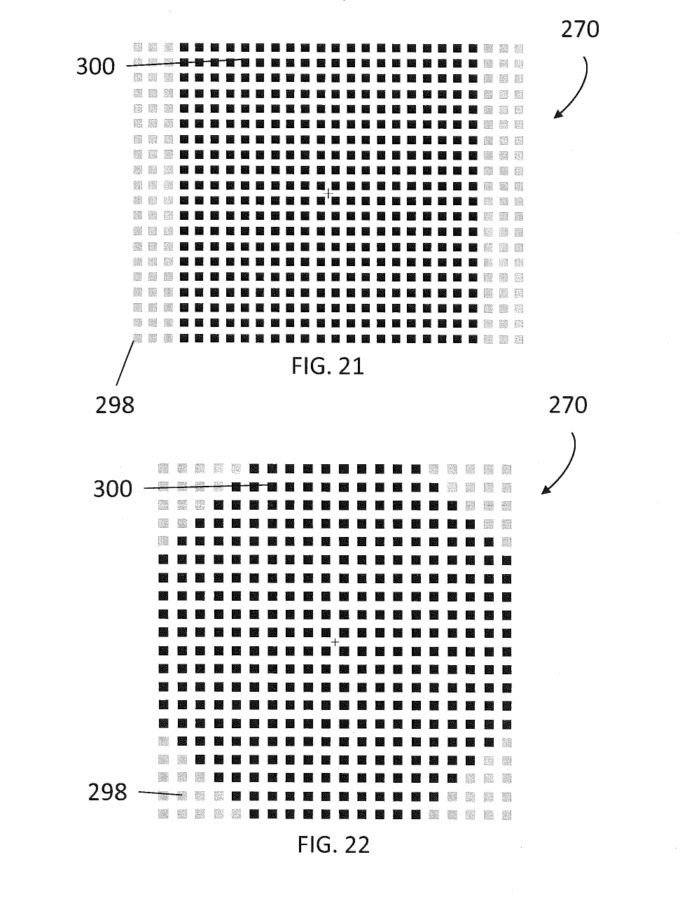

In some aspects, the ground electrodes 298 arranged about the periphery of the transducer array 270 may be much less than the number of transducer elements 272 (and thus the corresponding number of signal electrodes 300) in the array 270. For example, a 20×20 transducer array with 125 μm pitch may yield a transducer array of about 2.5 mm in width. In such an instance, a catheter size of 10 French (2.8 mm I.D.) would be needed. Thus, only one ring of ground electrodes 298 could be disposed about the periphery of the transducer array, resulting in a 22×22 array with an overall width of about 2.75 mm. As such, the arrangement would include 400 transducer elements 272 (corresponding to 400signal electrodes 300 disposed within the periphery) and 84 ground electrodes 298. If corresponding connective signal and ground elements 150, 160 are incorporated into the corresponding cable assembly 325, the relatively few connective ground elements 160 may not necessarily provide adequate shielding for the connective signal elements 150. As such, further aspects of the present disclosure are directed to other arrangements, whether actual or constructive, wherein the connective ground elements 298 of the cable assembly 325 are alternatingly disposed or otherwise interspersed with respect to the connective signal elements 150 along the length of the cable assembly 325. One skilled in the art will appreciate that other arrangements may be provided in order to increase the ratio of ground to signal wires without increasing one or both lateral dimensions of the transducer array. For example, as shown in FIG. 21, additional columns of connective ground elements 298 may be arranged only along one axis of the transducer array 270 adjacent to the connective signal elements 300. More particularly, such an arrangement may include, for example, a 20×26 array containing 400 connective signal elements and 120 connective ground elements, or a 20×40 array containing 400 connective signal elements and 400 connective ground elements. In another aspect, for instance, the corners of the array can be implemented as connective ground elements 298 in order to maintain array size along both cross-sectional axes. More particularly, for example, the 20×20 array shown in FIG. 22 may be configured to include 340 connective signal elements and 60 connective ground elements.

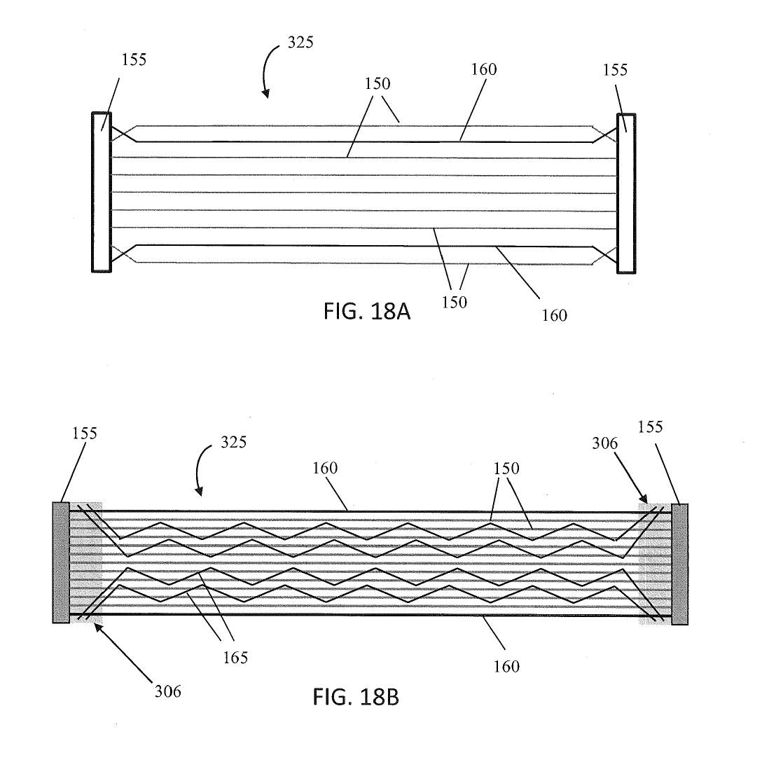

In this regard, as shown in FIG. 18A, the connective signal elements 150 and connective ground elements 160 may be intermingled such that the connective ground elements are substantially or constructively alternatingly disposed or otherwise interspersed with respect to the connective signal elements 150, regardless of the position in which the respective connective ground element 160 interacts with the termination element (i.e., connection support substrate 155). That is, in instances where the connective ground elements engage the termination element (i.e., connection support substrate 155) about the periphery thereof, the respective connective ground elements 160 may be routed between the connective signal elements 150 at least partially along the length of the cable assembly 325 (i.e., from the periphery about the first termination element, along the cable assembly 325 at an interstitial site, and back to the periphery about the opposing second termination element). Further, in some instances, the connective signal elements 150 and connective ground elements 160 may be twisted (i.e., in pairs or larger number of such elements) together, at least partially along the length of the cable assembly 325, as another manner of routing the connective ground elements 160 between the connective signal elements 150 at least partially along the length of the cable assembly 325. Further, as shown in FIG. 18B, additional connective ground elements 165 may be attached or otherwise incorporated in to the cable assembly 325 using, for example, a conductive epoxy 306 applied to the support substrate 155 and/or otherwise to the cable assembly 325, wherein such additional connective ground elements 165 may be further interspersed between the connective signal elements 150. Such additional connective ground elements 165 may further be inserted into the connection support substrate 155, or may be separate elements, externally-attached using, for example, a conductive epoxy material. The additional connective ground elements 165 may be, for instance, individual wires, strips of metal foil, and/or other conductive material interspersed between the connective signal elements (i.e., wires) to provide additional shielding therefor.

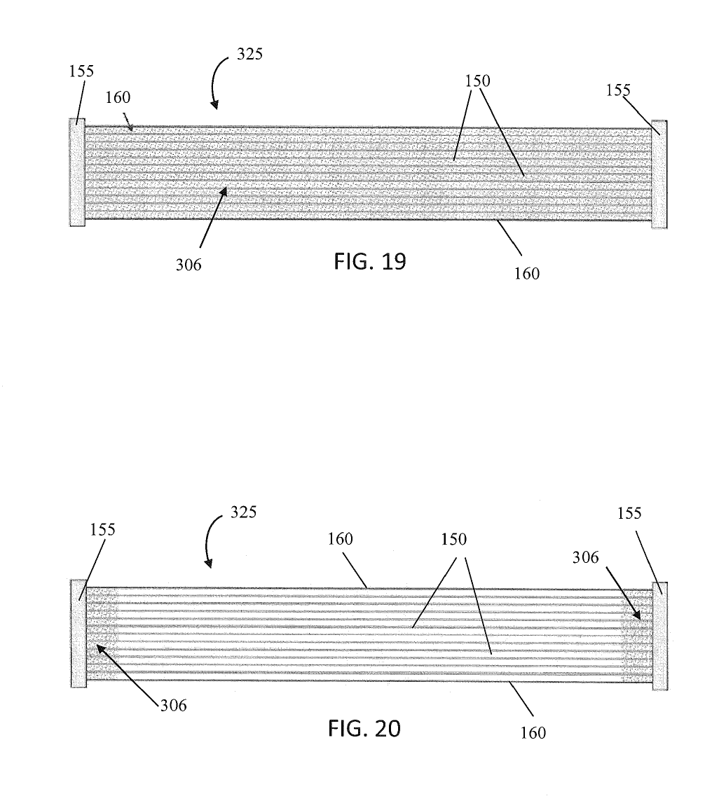

In another aspect, as shown in FIG. 19, the connective signal elements 150 may be insulated along the length of the cable assembly 325, while the connective ground elements may be bare, or at least partially bare, conductive material (i.e., bare or partially bare copper wire). In such instances, a conductive epoxy material 306 may be applied to the connective elements 150, 160 so as to extend between the collective elements 150, 160 and collectively encapsulate the connective elements 150, 160. The conductive epoxy material 306 may extend along the cable assembly 325 between the opposed ends, as shown in FIG. 19, or only partially along the length of the cable assembly 325, as shown in FIG. 20. The conductive epoxy material 306 may be, for example, a silicone, a urethane-based epoxy or other flexible epoxy filled with conductive particles or otherwise having conductive particles incorporated therein, or other suitable material, wherein such epoxy materials may promote or facilitate flexibility of the cable assembly 325. Further, such a conductive epoxy material 306 may form an electrically-conductive engagement with the connective ground elements (bare or partially bare conductive material) 160 so as to essentially form a single conductive body extending about and between all connective signal elements 150. Such a configuration thus constructively facilitates connective ground elements 160 that are alternatingly disposed with respect to the connective signal elements 150 along the cable assembly 325.

According to another aspect, the connective signal elements 150 may be coated with a conductive coating material applied to each such elongate insulated element extending along the cable assembly 325. In such instances, the connective ground elements 160 may be at least partially in electrically-conductive communication with the connective signal elements 150 via the conductive coating material, thus also constructively facilitating connective ground elements 160 that are alternatingly disposed with respect to the connective signal elements 150 along the cable assembly 325. For example, a conformal thin film copper layer may be deposited on the insulator material covering the connective signal elements 150 by metal organic chemical vapor deposition (MOCVD), electroless plating, or a conductive spray process. Such a coating may form a coaxial conductor configuration for each connective signal element 150, such that this outer coating may be electrically-connected to the connective ground elements 160 via the conductive epoxy 306 applied thereto, thus providing additional shielding around each connective signal element 150. Coating the connective signal elements 150 with a conductive substance may further facilitate increased flexibility of the cable assembly 325 by allowing the conductive epoxy material 306 to be applied only partially along the length of the cable assembly 325, as shown in FIG. 20. In such an aspect, the conductive epoxy material 306 may be applied to the connective elements 150, 160 proximate to the connection support substrates 155 and not along the entire length of the cable assembly 325. Such a configuration constructively facilitates connective ground elements 160 that are alternatingly disposed with respect to the connective signal elements 150 about the connection support substrates 155 by way of the conductive epoxy material 306, while such alternating disposition of the connective ground elements 160 is otherwise facilitated by the conductive coating material applied to the connective signal elements 150 (i.e., by way of conductive physical contact between the conductive coating material and the bare conductive material of the conductive ground elements 160) along the length of the cable assembly 325 free of the conductive epoxy material 306.

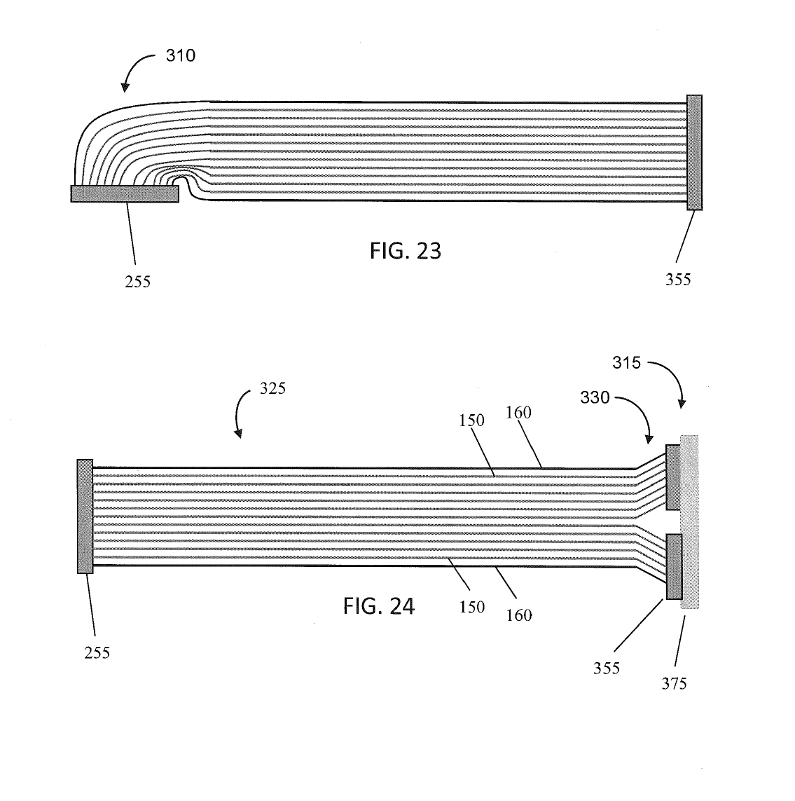

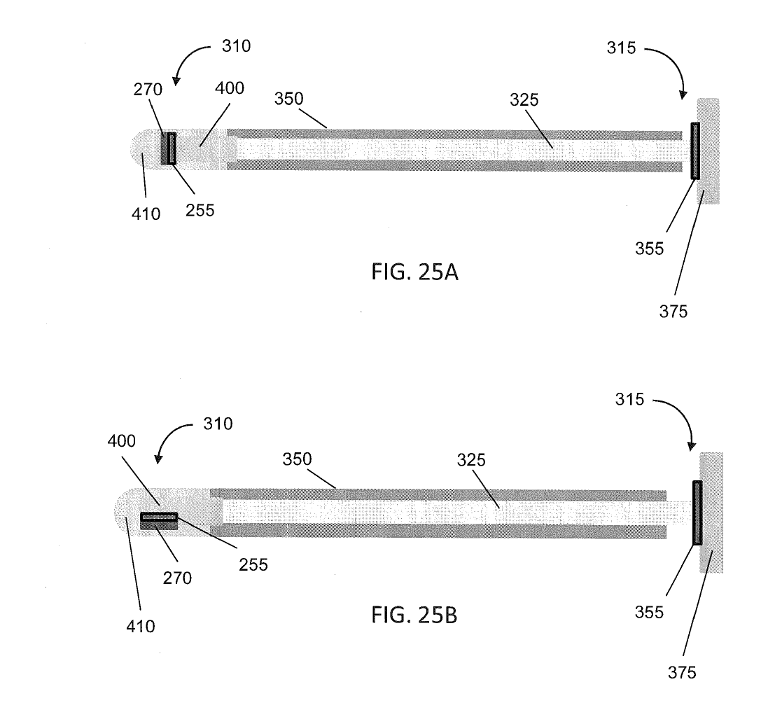

The cable assemblies shown in FIGS. 16-20 are exemplary cable assemblies for forward-looking 1D or 2D arrays as shown, for instance, in FIG. 14. In another aspect, similar cable assemblies can be configured, for example, as shown in FIG. 23, for side-looking 1D and 2D arrays, as shown in FIG. 15. In such instances, the connection support substrate 255 bonded to or otherwise engaged with the transducer array 270 may be configured to facilitate a change in direction of the connective signal and ground elements 150, 160 extending longitudinally along the catheter to a mating arrangement for the transducer array 270, wherein such a mating arrangement may be oriented perpendicularly to the longitudinal axis of the catheter. Once engaged with the mating arrangement, the signal and ground wires (the connective signal and ground elements) can then be bent approximately 90 degrees to extend the connective elements substantially parallel with the longitudinal axis of the catheter. Such a configuration of the cable assembly may thus also facilitate bending of the connective signal and ground elements, for example, in comparison to a multiple level flex cable arrangement that may be relatively stiffer and more difficult to bend without risk of damage to the flex cable assembly. For the assembly shown in FIG. 23, each individual conductor wire (conductive element) may have a relatively small diameter (e.g., between about 40 AWG and about 50 AWG), and may thus be relatively flexible and readily bent at about a 90 degree angle. The bent conductors may then be, for example, encapsulated by an epoxy material such as, for example, a potting epoxy 400, as shown in FIG. 25B, to provide stiffness and/or strain relief for the conductors, adjacent to the connection support substrate 255 attached to the transducer array 270 disposed at the distal end 310 of the catheter member 350.

In some instances, the distal end 310 of the catheter member 350 may also include a fluid-containing or fluid-filled capsular member 410, as shown in FIGS. 25A and 25B, configured to house at least the pMUT device 270. The fluid contained within the capsular member 410 may, for example, facilitate acoustic transmission of the acoustic energy emitted by the pMUT device 270 through the catheter wall and into the body or fluid bed of the organ being imaged, for example, the heart or a vessel. Some standard or existing piezoelectric ultrasound transducers may be embedded in an epoxy material to facilitate acoustic transmission via an epoxy matching layer. However, pMUT devices according to aspects of the present disclosure include flexible transducer membranes (i.e., piezoelectric material 278) that are preferably configured and arranged to avoid mechanically loading or constraint by a mechanical impediment such as an epoxy layer. Thus, the fluid medium contained within the capsular member 410 and in contact with the pMUT transducer array 270 may provide an advantageous configuration for improve signal transmission and imaging capabilities. The fluid contained within the capsular member 410 may comprise, for instance, silicone or other fluid with appropriate viscosity, for example, between about 1 cSt and about 100 cSt, and/or appropriate acoustic impedance, for example, between about 1 MRayl and about 1.5 MRayl, or less than about 5 MRayl. Once formed, the pMUT device 270 having the connection support substrate 255 engaged therewith may be inserted into the lumen of the catheter and, in turn, into the capsular member 410 disposed about the distal end 310 of the catheter. In other aspects, the capsular member 410 may be engaged with the distal end 310 of the catheter, externally or substantially externally to the lumen of the catheter. The capsular member 410 may then be filled or substantially filled with the appropriate fluid, and the capsular member then be sealed, whether about the cable assembly 325 or otherwise, using, for example, heat or epoxy, to form a fluid tight seal. In some aspects the capsular member 410 may be sealed to as to contain at least the pMUT device 270. However, in some instances, the capsular member 410 may be sealed about the connection support substrate 255 engaged with the pMUT device 270, about the epoxy material (i.e., potting epoxy 400), if present, applied to the connective elements of the cable assembly 325 adjacent to the connection support substrate 255, or about the cable assembly 325 itself.

At the proximal end 315 of the catheter 350, the connection support substrate 355 of the cable assembly 325 may be engaged with or otherwise terminated by a termination element 375, such as, for example, an interposer, circuit board or semiconductor package. In this regard, the distal end connection support substrate 255 may have a pitch of the connective signal and ground elements 150, 160 that is approximately the same as the transducer array 270 in order to facilitate bonding and electrical engagement of the connective elements to the pMUT array 270. Such a relatively fine pitch may also facilitate extension of the connective elements substantially parallel (or first bent at about 90 degrees and then extension substantially parallel) to the longitudinal axis of the catheter in a close-packed configuration. Such an arrangement may, for instance, allow several hundred conductors to fit within a small, for example, 3 mm diameter, catheter 350. About the proximal end 315 of the catheter 350, the connective signal and ground elements 150, 160 engaged with the connection support substrate 355 may be configured to electrically-engage corresponding conductor elements associated with a termination element 375 such as, for example, an interposer, circuit board or semiconductor package. Such conductor elements may comprise, for example, metal conductors deposited by electroplating, RF sputtering or evaporation, and patterned on the surface of the termination element 375. The conductor elements of the termination element 375 may, for example, facilitate an electrically-conductive engagement between the connective signal and ground elements 150, 160 associated with the connection support substrate 355, and, for instance, a connector cable for the ultrasound system, solder bumps attaching additional circuitry by flip chip bumping, or other devices configured to facilitate generation of the ultrasound image by an external device or system. In another aspect, such an arrangement may be advantageous, for example, by providing a cable assembly 325 having a relatively lower materials cost. For example, insulated magnet wire may cost approximately $0.004 per meter length, whereas some flex cables containing 16 conductors may cost approximately $10 per meter length. Thus, for 256 conductors in a 1 meter length catheter, magnet wire may cost about $1 per catheter, whereas flex cable could cost about $160 per catheter. Such an example thus illustrates the magnitude of the cost savings that may be realized according to various aspects of the present disclosure.

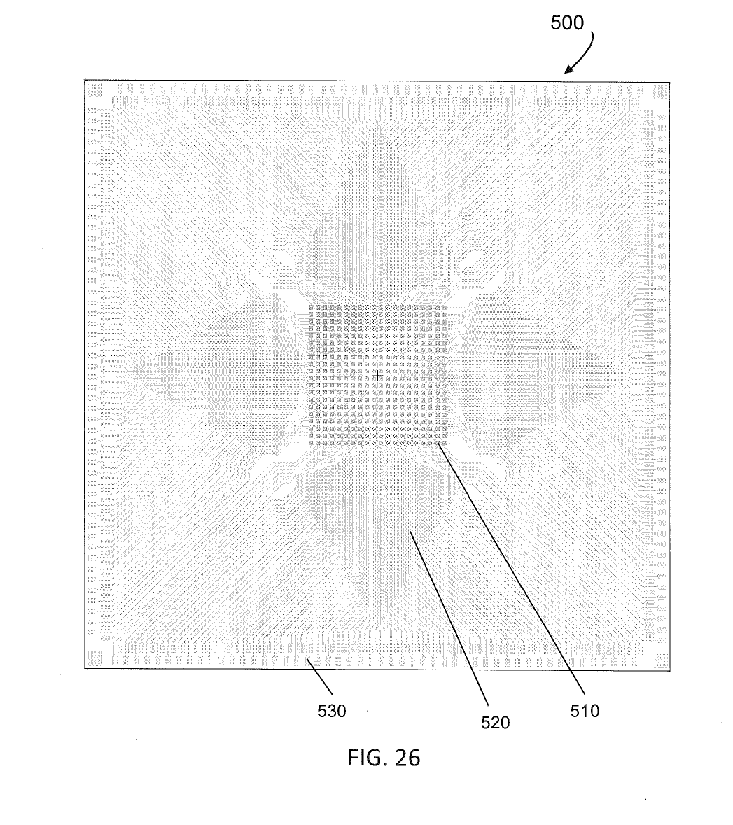

In some instances, the pitch of the connective signal and ground elements 150, 160 may be increased in order to facilitate engagement with the termination element 375 and, in turn, engagement between the termination element 375 and the external ultrasound system. For example, routing 400 connective signal elements (20×20 array) with respect to an interposer device having an element pitch of between about 100 microns and about 200 microns may be difficult without requiring conductor traces on the interposer device to be extremely narrow and close together. Such a configuration could undesirably cause cross talk between conductor traces, as well as increased ohmic resistance thereof, which could degrade the signals carried thereby. An example of such a termination interposer device 500 is shown, for example, in FIG. 26, wherein such an interposer device 500 includes a 20×20 array of signal pads 510 for engaging the connective signal elements 150. Such signal pads 510 are routed through signal traces 520 to connection pads 530, wherein the interposer device can then be electrically-connected through the connection pads 530, via wirebonding or solder bumping, for example, to a circuit board or other semiconductor package. In instances where the spacing between the signal pads 510 is about 75 μm, the pitch of the signal traces 520 may be as small as about 16 μm, with the width of the signal traces 520 being as small as about 8 μm, and the length of the signal traces 520 being at least several millimeters from the signal pads 510 to the connection pads 530 disposed about the edges of the interposer device. The 20×20 array may be about 4 mm in width, while the interposer device may have a width of about 17 mm. Further, as many as 4 signal traces 520 may be routed between signal pads 510. Such a relatively fine pitch and relatively narrow trace width could thus carry the risk of causing signal degradation.

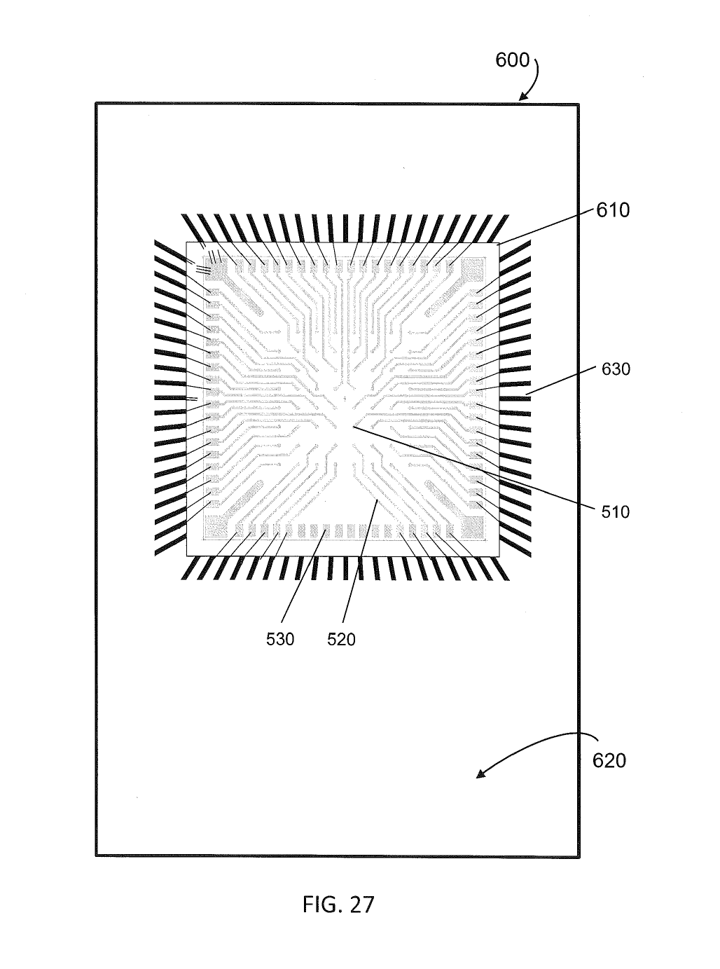

As such, according to some aspects, an arrangement for termination of the connection support substrate 355 is shown, for example, in FIGS. 24 and 27. In such aspects, the main cable assembly 325, containing several hundred conductors, can be divided into smaller cable subassemblies 330 near the proximal end 315, as shown, for example, in FIG. 24. For example, a cable assembly 325 with 600 conductors can be divided into 8 subassemblies 330 containing 75 conductors each, with each subassembly 330 toward the proximal end 315 having its own termination connection support substrate 355. Each subassembly 330 may be bonded to an individual interposer device 610 configured as shown, for example, in FIG. 27. As shown in FIG. 27, a termination circuit board 600 may include routing to connect termination interposer devices 610 to connectors 620 for a cable extending to the external ultrasound system. For example, for a cable assembly 325 having 512 signal wires (connective signal elements), each termination interposer device 610 may include, for example, 64 signal traces. One or more interposer devices 610 can be connected to connection pads 630 on the termination circuit board 600, for instance, via wirebonding, by solder bumping, or by mounting the interposer devices 610 into semiconductor packages (not shown) that are connected to the termination circuit board 600. The routing associated with the termination circuit board 600 may then be implemented to electrically engage the signal traces to the connector 620 associated with the external ultrasound system. In such instances, signal trace routing associated with the interposer device 610 can be accomplished with relatively shorter traces, relatively wider signal traces, and relatively larger pitch, thus reducing or otherwise eliminating signal degradation.

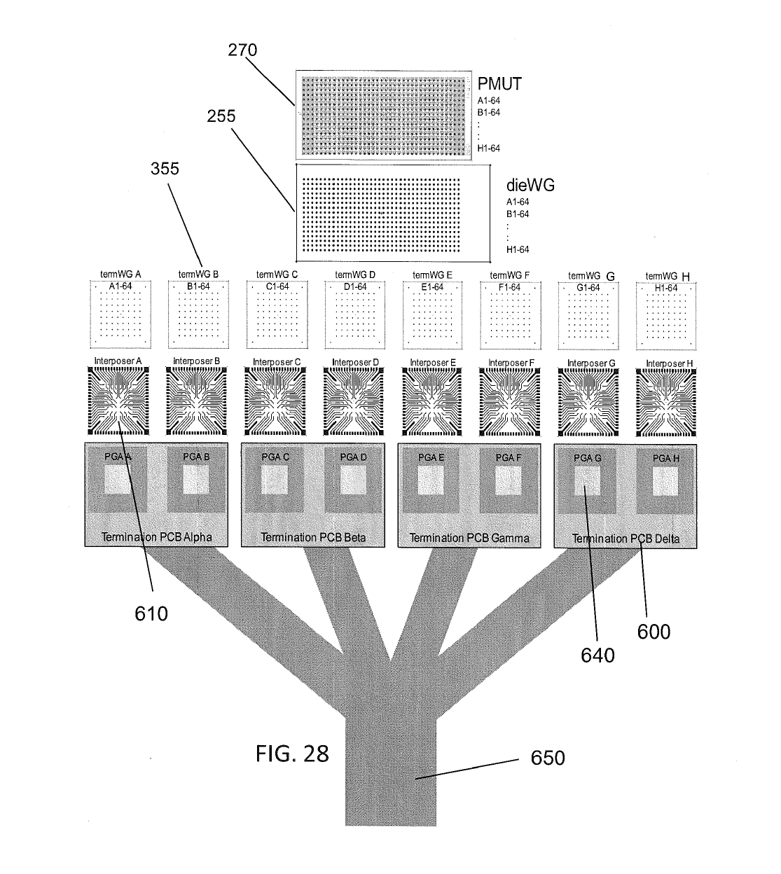

An exemplary overall schematic of a component layout for a pMUT array 270 with associated cable assembly and termination element is shown in FIG. 28. The transducer array 270 may be bonded to the distal connection support substrate 255 having the connective signal and ground elements 150, 160 (not shown) engaged therewith using, for example, solder bumps, gold stud bumps or an anisotropic conductive epoxy, to provide an electrically-conductive connection between the pMUT array elements and the connective signal and ground elements (wires) 150, 160. The connective elements extend as part of the cable assembly (not shown) to the proximal connection support substrate(s) 355 (i.e., the termination). The connection support substrates 355 may then be bonded to the termination interposer devices 610 using, for example, solder bumps, gold stud bumps or an anisotropic conductive epoxy, to provide an electrically-conductive connection between the connective signal and ground elements (wires) 150, 160, and the signal pads 510 of the respective interposer device 610. The interposer routing 520 subsequently provides an electrically-conductive connection to the connection pads 530 of that interposer device 610, which may then, in turn, be wirebonded or otherwise electrically connected to the connection pads 630 of a termination PC board 600 associated with the external ultrasound system. In other aspects, the interposer devices 610 may be wirebonded into semiconductor packages 640 that may be mounted onto termination PC boards 600 using, for example, electrically conductive pins or solder bumps. In still other aspects, the interposer devices 610 may instead include through-silicon vias or through-substrate vias substantially filled with a conductive material, wherein such vias, or other conductive traces associated with the interposer device 610, may be attached to connection pads 530 on the termination PC board 600, for instance, via solder bumps, gold stud bumps, or an anisotropic conductive epoxy. The termination PC board 600 further routes the connections with the connective elements from the interposer device to the connector 620 associated with a cable 650 extending to the external ultrasound system. In some instances, the termination PC board 600 may also include other circuitry to facilitate forming of an ultrasound image, for example, transmit pulsers, transmit beamformers, amplifiers, receive beamformers, transmit/receive switches, timing circuits, and other appropriate circuitry and/or components, as will be appreciate by one skilled in the art.

Aspects of a cable assembly 325 as disclosed herein may be implemented, in some instances, with other types of appropriately-configured ultrasound transducers, as will be appreciated by one skilled in the art. Such an appropriately configured ultrasound transducer may comprise, for example, a PZT ceramic ultrasound transducer with signal and/or ground electrodes on at least one side of the transducer array for connection to a connection support substrate of the cable assembly 325. In another aspect, such an ultrasound transducer may comprise, for instance, a capacitive micromachined ultrasound transducer (cMUT), that may include through-silicon or through-substrate vias for providing electrically-conductive connections with the back side of the substrate, may be bonded to a connection support substrate of the cable assembly 325. Thus, a cable assembly 325 according to aspects of the present disclosure may be implemented with many other types and configurations of ultrasound transducers to facilitate the connection of a relatively large number of connective signal and ground elements in a relatively small diameter probe, such as a catheter or endoprobe. In some exemplary instances, pMUT arrays or other transducer arrays assembled with such cable assemblies may be advantageous in catheters or other probes having a relatively small diameter and a relatively high number of transducer elements, for instance, for use in interventional cardiology or interventional radiology applications, such as intravascular or intracardiac surgical procedures. In other instances, such transducers and cable assemblies may be advantageous in other types of endoprobe devices having a relatively small diameter and a relatively high number of transducer elements, such as laparoscopic ultrasound probes used for minimally invasive surgeries, such as prostate, liver or gall bladder procedures.

Many modifications and other aspects of the disclosures set forth herein will come to mind to one skilled in the art to which these disclosures pertain having the benefit of the teachings presented in the foregoing descriptions and the associated drawings. For example, one such aspect of an ultrasound transducer with associated cable assembly may be provided in instances where the transducer and cable assembly are configured for use in a side-looking intracardiac catheter, having an outer diameter of about 14 French (about 4.6 mm). More particularly, a pMUT array may be fabricated with 512 transducer (pMUT) elements 272 (i.e., a 16×32 array) and 96 ground pads or electrodes 298, generally configured as shown, for example, in FIG. 4. As disclosed, the pMUT array may be configured such that the structure of the pMUT elements 272 includes through-substrate vias/interconnects, so as to provide electrically-conductive connections to the ground and signal electrodes 298, 300 of the pMUT elements 272 within the array on the back side of the substrate. The pitch of the pMUT elements in the pMUT array may be on the order of about 175 μm for an overall array size of about 2.8 mm×5.6 mm. The 2.8 mm array width is configured to fit within the lumen of a 14 French catheter (inner diameter of about 3.8 mm). Such a pMUT array is thus configured to be capable of real-time 3D ultrasound imaging. As a result of such a configuration, one signal conductor is required per pMUT element in the pMUT array in order to allow the pMUT elements to be individually actuated. However, this arrangement results in a relatively higher number of required conductors in the cable assembly than conventional flex cable, micro-coax cable or micro-ribbon cable can provide in a sufficiently small form factor for implementation in an intracardiac catheter. Conventional 2D linear array devices used in ultrasound catheters typically include only 64 transducer elements; therefore, conventional cabling can be used in such instances.

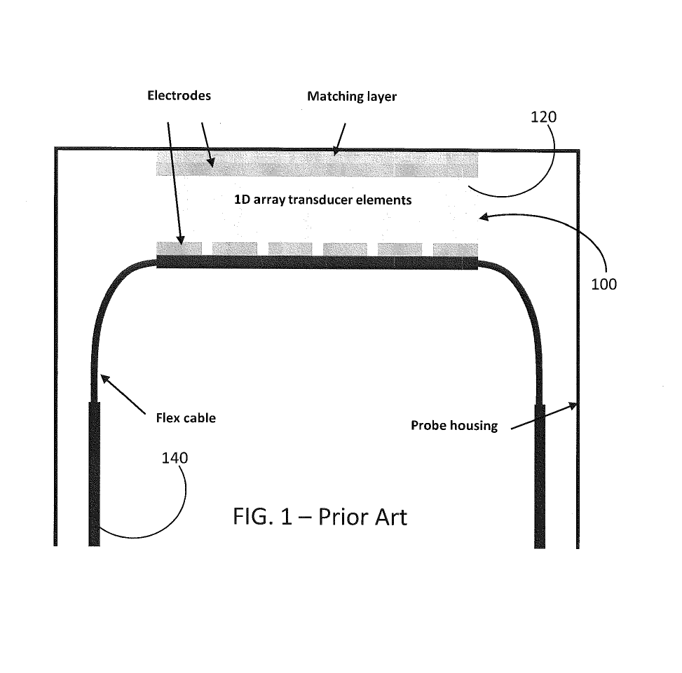

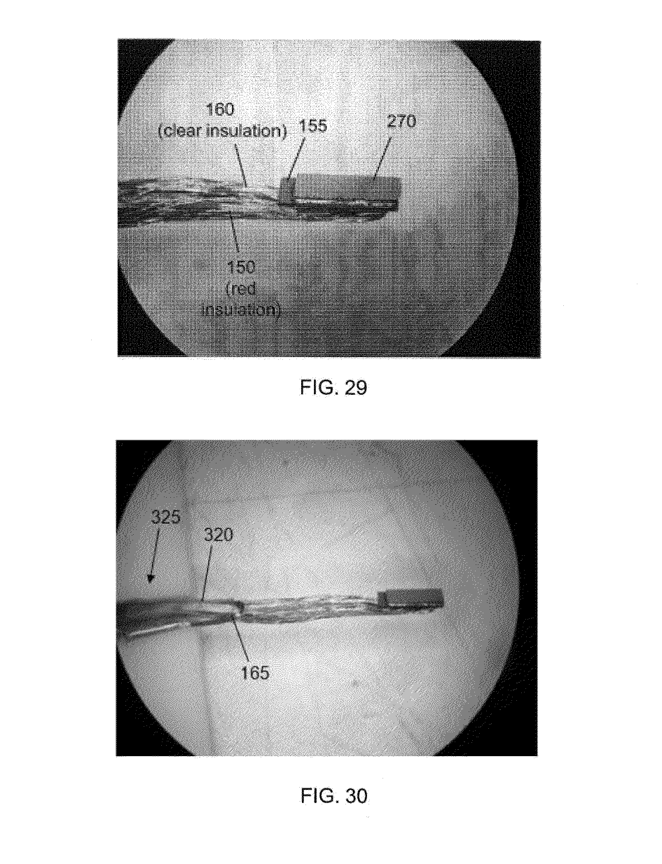

Accordingly, in order to overcome the noted limitations of conventional cables, while meeting the noted requirements, one exemplary aspect may be directed to a cable assembly, as shown in FIG. 29, including on the order of 512 connective signal elements and 128 connective ground elements. However, in some instances, the cable assembly may include at least 100 connective signal elements though, in other instances, the cable assembly may include at least 400 connective signal elements, consistent with the principles discussed in relation to the these and other aspects of the disclosure. In such an aspect, the connection support substrate 155 may be comprised, for example, of silicon or any other suitable material, in which vias may be etched therethrough using, for instance, a DRIE process. The connective signal and ground elements may then be directed/inserted into the etched vias in a connection support substrate in correlation with the pattern of transducer elements and ground pads in the pMUT array. The connective signal and ground elements may be on the order of between about 40 AWG and about 50 AWG in diameter, wherein, in one instance, the connective signal and ground elements may be 45 AWG insulated magnet wire. For differentiation/distinction during formation of the cable assembly, the connective signal elements 150 may be configured to have red insulation, while the connective ground elements 160 may be configured to have clear, white, or any other suitable color insulation distinguishable from the insulation of the connective signal elements. In some instances, the pitch of the vias/through-holes in the connection support substrate may be on the order of less than 200 microns though, in other instances, the pitch may be on the order of less than about 100 microns. In one particular aspect, the pitch about 175 μm to correspond to the pitch of the connections for the pMUT array 270 previously disclosed. The connective signal and ground elements may be secured in the vias of the connection support substrate using, for example, a low-viscosity insulating epoxy material or any other suitable bonding material. In any instance, the surface of the connection support substrate configured to engage the pMUT array 270 may first be polished to expose the ends of the connective signal and ground elements extending therethrough and to provide a flat surface for bonding to the pMUT array. The pMUT array and connection support substrate may be bonded together, for example, using an epoxy material, wherein the exposed ends of the connective signal and ground elements are in electrically-conductive engagement with the signal and ground contacts on the back side of the pMUT array. FIG. 29 shows one example of a pMUT array 270 bonded to a connection support substrate 155 with associated connective signal and ground elements (i.e., wires) 150, 160, as previously disclosed. The wires, proximate to the ends thereof, may also be individually bent at an angle of about 90 degrees to provide a side-looking catheter configuration as shown, for example, in FIG. 23. Because the cable is made of individual wires, there may be little or no mechanical stress on the conductors, compared to bending a flex cable assembly (e.g., as shown in FIG. 1), wherein such bending may impart significant stress on the collective flex cable assembly.

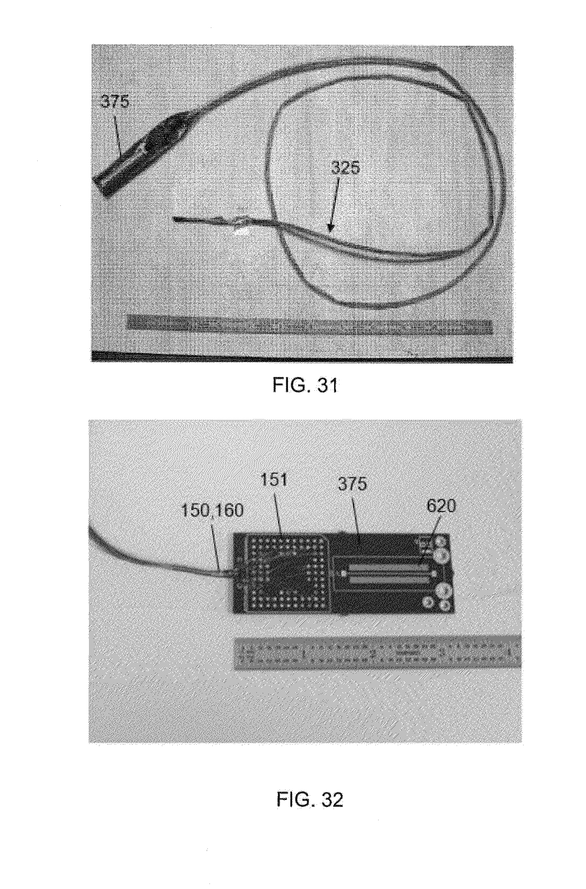

The connective ground elements may be connected to the ground contacts of the pMUT array laterally outward of the connective signal elements as shown, for example, in FIG. 21. Additional connective ground elements (wires) 165 may be provided in the cable assembly over the number of available ground contacts in the pMUT array, for example, to provide additional shielding of the connective signal elements, as shown, for instance, in FIG. 30 (see also, e.g., FIG. 18B), wherein in one aspect, a total of 128 ground wires may be provided in the cable assembly for connection to the ground contacts of the pMUT array and to shield the 512 connective signal elements in the cable assembly. In such instances, for example, every 4 connective signal elements (wires) may be twisted together with one connective ground element (wire) in the cable assembly to facilitate shielding of the connective signal elements by interspersing the connective ground elements among the connective signal elements. Sheathing, such as shrink tubing 320 may be provided and installed about the connective elements of the cable assembly so as to provide a more robust sheathed cable assembly 325. In some aspects, the connective elements of the cable assembly may be terminated opposite to the connection support substrate by a termination element such as, for example, a printed circuit board (PCB) 375 as shown in FIG. 31. The PCB itself may also include a connector 620 for forming an electrically-conductive connection with an ultrasound system. The conductive signal and ground elements 150, 160 may be engaged (i.e., soldered) with conductively-plated vias 151 in the PCB 375 as shown, for example, in FIG. 32. There may be, for example, between one and eight PCB's engaged with the free ends of the connective signal and ground elements of the cable assembly, opposite to the connection support substrate, as necessary or desired, depending, for instance, on the number of connective signal and ground elements in the cable assembly and the number of pinouts on the respective PCB, though the number of PCB's may vary considerably.

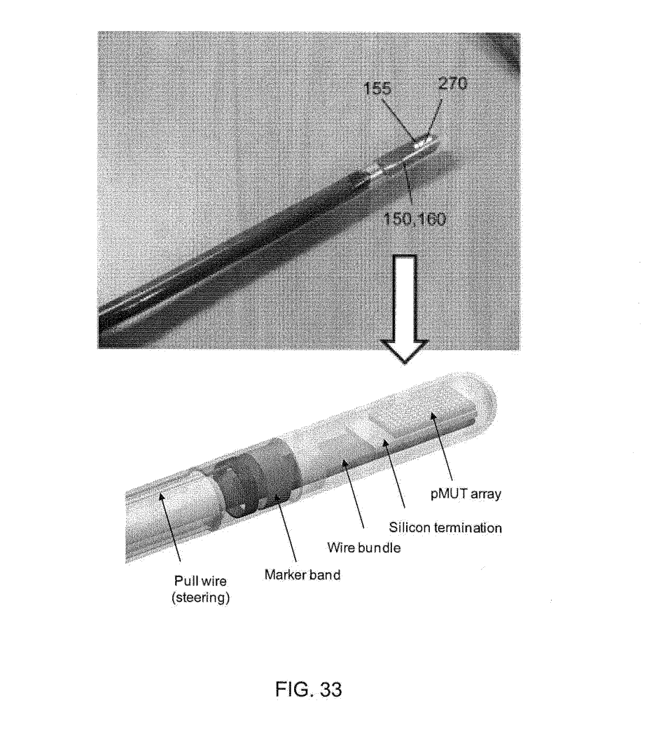

The cable assembly 325 shown, for example, in FIG. 31, may have a length of about 50″ for implementation in a 36″ length intracardiac catheter. The distal end of a 14 French catheter assembly is shown, for example, in FIG. 33, with the transducer array 270, connection support substrate 155 and connective signal and ground elements 150, 160 visible in the distal tip of the catheter assembly. The catheter housing may be comprised of Pebax® with embedded metal braiding. In some instances, the catheter housing may include a marker band for facilitating visualization of the tip under fluoroscopy and/or a pull wire to deflect the catheter tip (i.e., catheter control/steering). In particular aspects, the catheter assembly may be particularly configured for real-time 3D intracardiac ultrasound imaging. For example, the catheter assembly may be placed in the right atrium via the inferior vena cava for imaging an ablation catheter in the left atrium during a pulmonary ablation procedure.

Other examples of transducer and cable assemblies, such as disclosed in the present aspect, may be used for intravascular imaging. In such instances, the catheter assembly may be required to have a relatively smaller outer diameter, for example, of no more than about 6 French (about 2 mm). In order to meet the size constraints of the catheter assembly, the transducer array may have fewer elements and, as such, the corresponding cable assembly may have fewer signal wires that must fit within the inner diameter of the catheter. For example, in such instances, the size constraint may be met by a transducer array of 256 pMUT elements (16×16 transducer array), with a pMUT element pitch of about 60 microns, and with the cable assembly including 256 connective signal elements and 64 connective ground elements. In such a configuration, the connection support substrate would require a via pitch of about 60 microns to correspond with the signal and ground contact pitch of the transducer array, so as to facilitate an electrically-conductive engagement therebetween. In some instances, the connective signal and/or ground elements (wires) may be configured with a relatively smaller diameter, e.g., between about 45 AWG and about 50 AWG, so as to reduce, or further reduce, the lateral dimension of the cable assembly. Such an intravascular catheter could be used, for example, for real-time 3D ultrasound imaging of a stent placed in an artery or for imaging an occlusion in an artery. Accordingly, such a catheter assembly may be appropriately scaled, for example, so as to be configured to fit within a 2 mm catheter (i.e., with >100 connective signal elements at a pitch of <100 μm for intravascular ultrasound applications), or to fit within a 3-4 mm catheter (i.e., with >400 connective signal elements at a pitch of <200 μm for intracardiac echo applications).

Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. An ultrasound device, comprising:
an ultrasonic transducer device comprising a plurality of transducer elements forming a transducer array, each transducer element comprising a piezoelectric material disposed between a first electrode and a second electrode, one of the first and second electrodes comprising a ground electrode and the other of the first and second electrodes comprising a signal electrode; and
a cable assembly comprising a plurality of connective signal elements and a plurality of connective ground elements extending in substantially parallel relation therealong, each being configured to form an electrically-conductive engagement with respective ones of the signal electrodes and the ground electrodes of the transducer elements in the transducer array, the connective ground elements being configured to be alternatingly disposed with the connective signal elements across the cable assembly, to provide shielding between the connective signal elements.

2. An ultrasound device according to claim 1, further comprising a connection support substrate disposed about at least one end of the cable assembly and configured to receive the connective signal elements and the connective ground elements of the cable assembly therethrough.

3. An ultrasound device according to claim 2, wherein a first connection support substrate is configured to be engageable with the ultrasonic transducer device so as to form the electrically-conductive engagement between the connective signal elements and connective ground elements and the respective ones of the signal electrodes and the ground electrodes.

4. An ultrasound device according to claim 3, wherein a second connection support substrate is configured to be engageable with one of an interposer device and a termination element.

5. An ultrasound device according to claim 2, further comprising at least one printed circuit board engaged with the connective signal elements and the connective ground elements opposite the connection support substrate.

6. An ultrasound device according to claim 2, wherein at least one end of the cable assembly comprises one of a plurality of connection support substrates and a plurality of termination elements engaged therewith and in communication with the connective signal elements and the connective ground elements, each of the plurality of connection support substrates being configured to be engageable with one of an interposer device and a termination element.

7. An ultrasound device according to claim 4, wherein the interposer device comprises at least two conductors, each conductor having opposed first and second ends, and configured to form electrically-conductive engagements with the connective signal elements and the connective ground elements, via the other of the connection support substrates.

8. An ultrasound device according to claim 1, wherein at least one of the connective signal elements and at least one of the connective ground elements of the cable assembly are twisted together to provide shielding between the connective signal elements.

9. An ultrasound device, according to claim 1, further comprising a conductive epoxy material in electrically-conductive engagement between the connective ground elements, and extending between the connective signal elements, so as to provide shielding between the connective signal elements.

10. An ultrasound device according to claim 2, wherein at least one of the ends of the cable assembly includes an epoxy material applied about the connective signal elements and the connective ground elements adjacent to the corresponding connection support substrate.

11. An ultrasound device according to claim 9, wherein the conductive epoxy material extends at least partially along the cable assembly.

12. An ultrasound device according to claim 9, wherein the conductive epoxy material comprises a flexible epoxy material having conductive particles incorporated therein.

13. An ultrasound device according to claim 1, wherein the connective signal elements comprise elongate insulated elements and the connective ground elements comprise elongate uninsulated elements.

14. An ultrasound device according to claim 13, further comprising a conductive coating material applied to each elongate insulated element, the connective ground elements being at least partially in electrically-conductive communication between the connective signal elements via the conductive coating material.

15. An ultrasound device according to claim 14, wherein the conductive coating material comprises one of a conformal copper thin film coating, an electroless plating, and a conductive spray film

16. An ultrasound device according to claim 1, further comprising at least one external ground conductor arranged in an electrically-conductive engagement with the connective ground elements and extending therefrom to a ground.

17. An ultrasound device according to claim 16, wherein the at least one external ground conductor comprises one of a metal wire, a metal foil, and a conductive epoxy material.

18. An ultrasound device, comprising:
an ultrasonic transducer device comprising a plurality of transducer elements forming a transducer array, each transducer element comprising a piezoelectric material disposed between a first electrode and a second electrode, one of the first and second electrodes comprising a ground electrode and the other of the first and second electrodes comprising a signal electrode;
a catheter member having a distal end and defining a longitudinally-extending lumen, the lumen being configured to receive the ultrasonic transducer device about the distal end; and
a cable assembly comprising a plurality of connective signal elements and a plurality of connective ground elements extending in substantially parallel relation therealong, each being configured to form an electrically-conductive engagement with respective ones of the signal electrodes and the ground electrodes of the transducer elements in the transducer array, the connective ground elements being configured to be alternatingly disposed with the connective signal elements across the cable assembly such that the connective ground elements provide shielding between the connective signal elements.

19. An ultrasound device according to claim 18, further comprising a connection support substrate disposed about at least one end of the cable assembly and configured to receive the connective signal elements and the connective ground elements of the cable assembly therethrough.

20. An ultrasound device according to claim 19, wherein a first connection support substrate is configured to be engageable with the ultrasonic transducer device so as to form the electrically-conductive engagement between the connective signal elements and connective ground elements and the respective ones of the signal electrodes and the ground electrodes of the ultrasonic transducer device about the distal end of the catheter member.

21. An ultrasound device according to claim 20, wherein a second connection support substrate is configured to be engageable with one of an interposer device and a termination element away from the distal end.

22. An ultrasound device according to claim 19, further comprising at least one printed circuit board engaged with the connective signal elements and the connective ground elements opposite the connection support substrate.

23. An ultrasound device according to claim 19, wherein at least one end of the cable assembly comprises one of a plurality of connection support substrates and a plurality of termination elements engaged therewith and in communication with the connective signal elements and the connective ground elements, each of the plurality of connection support substrates being configured to be engageable with one of an interposer device and a termination element.

24. An ultrasound device according to claim 20, wherein the interposer device comprises at least two conductors, each conductor having opposed first and second ends, and configured to form electrically-conductive engagements with the connective signal elements and the connective ground elements, via the other of the connection support substrates.

25. An ultrasound device according to claim 18, wherein at least one of the connective signal elements and at least one of the connective ground elements of the cable assembly are twisted together to provide shielding between the connective signal elements.

26. An ultrasound device, according to claim 18, further comprising a conductive epoxy material in electrically-conductive engagement between the connective ground elements, and extending between the connective signal elements, so as to provide shielding between the connective signal elements.

27. An ultrasound device according to claim 19, wherein at least one of the ends of the cable assembly includes an epoxy material applied about the connective signal elements and the connective ground elements adjacent to the corresponding connection support substrate.

28. An ultrasound device according to claim 26, wherein the conductive epoxy material extends at least partially along the cable assembly.

29. An ultrasound device according to claim 26, wherein the conductive epoxy material comprises a flexible epoxy material having conductive particles incorporated therein.

30. An ultrasound device according to claim 18, wherein the connective signal elements comprise elongate insulated elements and the connective ground elements comprise elongate uninsulated elements.

31. An ultrasound device according to claim 30, further comprising a conductive coating material applied to each elongate insulated element, the connective ground elements being at least partially in electrically-conductive communication between the connective signal elements via the conductive coating material.

32. An ultrasound device according to claim 30, wherein the conductive coating material comprises one of a conformal copper thin film coating, an electroless plating, and a conductive spray film.

33. An ultrasound device according to claim 18, further comprising at least one external ground conductor arranged in an electrically-conductive engagement with the connective ground elements and extending therefrom to a ground.

34. An ultrasound device according to claim 33, wherein the at least one external ground conductor comprises one of a metal wire, a metal foil, and a conductive epoxy material.

35. An ultrasound device according to claim 18, further comprising a dielectric material collectively encapsulating the connective signal elements and the connective ground elements of the cable assembly.

36. An ultrasound device according to claim 35, wherein the dielectric material comprises one of a conformal dielectric coating and shrinkable tubing.

37. An ultrasound device according to claim 35, further comprising a conductive film collectively wrapped about the connective signal elements and the connective ground elements of the cable assembly, between the dielectric material and the connective signal elements and the connective ground elements, to provide shielding about the connective signal elements.

38. An ultrasound device according to claim 35, further comprising a conductive film wrapped about the dielectric material, between the catheter member and the dielectric material collectively encapsulating the connective signal elements and the connective ground elements, to provide shielding about the connective signal elements.

39. An ultrasound device according to claim 35, further comprising a conductive element incorporated into the catheter member so as to surround the dielectric material collectively encapsulating the connective signal elements and the connective ground elements, and to provide shielding about the connective signal elements.

40. An ultrasound device according to claim 18, further comprising a fluid-containing capsular member operably engaged with the distal end of the catheter member, the capsular member housing at least the ultrasonic transducer device.

41. An ultrasound device according to claim 27, further comprising a fluid-containing capsular member operably engaged with the distal end of the catheter member, the capsular member housing the ultrasonic transducer device, the corresponding connection support substrate engaged therewith, and the epoxy material applied about the connective signal elements and the connective ground elements adjacent to the connection support substrate.

42. A cable arrangement, comprising:
at least one connection support substrate; and
an elongate cable assembly having the at least one connection support substrate disposed about at least one end thereof, the cable assembly including a plurality of connective signal elements and a plurality of connective ground elements extending in substantially parallel relation therealong, each being configured to extend through the at least one connection support substrate and adapted to form an electrically-conductive engagement with respective ones of signal electrodes and ground electrodes of transducer elements in a transducer array, the connective ground elements being configured to be alternatingly disposed with the connective signal elements across the cable assembly such that the connective ground elements provide shielding between the connective signal elements.

43. A cable arrangement according to claim 42, wherein a first connection support substrate is disposed about a first end of the cable assembly and a second connection support substrate is disposed about a second end of the cable assembly.

44. A cable arrangement according to claim 42, further comprising at least one printed circuit board engaged with the connective signal elements and the connective ground elements opposite the at least one connection support substrate.

45. A cable arrangement according to claim 42, wherein the connective signal elements and connective ground elements each have a diameter of between about 40 AWG and about 50 AWG.

46. A cable arrangement according to claim 42, wherein the cable assembly includes at least 100 connective signal elements.

47. A cable arrangement according to claim 42, wherein the cable assembly includes at least 400 connective signal elements.

48. A cable arrangement according to claim 42, wherein the at least one connection support substrate is comprised of silicon and defines vias etched therein, the vias being configured to receive the connective signal elements and connective ground elements therein.

49. A cable arrangement according to claim 48, wherein the connective signal elements and connective ground elements are secured within the at least one connection support substrate using an insulating epoxy material.

50. A cable arrangement according to claim 48, wherein a pitch of the vias in the at least one connection support substrate is less than about 100 microns.

51. A cable arrangement according to claim 48, wherein a pitch of the vias in the at least one connection support substrate is less than about 200 microns.
